(19) United States
(12) Patent Application Publication
(10) Pub. No.: US 2020/0027526 A1
(43) Pub. Date: Jan. 23, 2020

(54) SYSTEMS AND METHODS FOR CREATION OF PERSONAL GENETIC PROFILE PRODUCTS

(71) Applicant: Orig3n, Inc., Boston, MA (US)

(72) Inventors: Robin Y. Smith, Boston, MA (US); Kate Blanchard, Boston, MA (US); Sunil Anant Gupta, Boston, MA (US); Edward Joseph Coffey, Boston, MA (US); Marcie A. Glicksman, Boston, MA (US); Brian Phillips, Boston, MA (US)

(21) Appl. No.: 16/471,187

(22) PCT Filed: Dec. 19, 2017
(86) PCT No.: PCT/US17/67264

§ 371 Date: Jun. 19, 2019

Related U.S. Application Data
(63) Continuation of application No. 15/445,752, filed on Feb. 28, 2017.
(60) Provisional application No. 62/485,322, filed on Apr. 13, 2017.
(60) Provisional application No. 62/436,947, filed on Dec. 20, 2016.
(60) Provisional application No. 62/436,947, filed on Dec. 20, 2016.

(51) Int. Cl. G16B 30/00; G16B 45/00; G16B 50/00; G16B 20/00

(52) U.S. Cl. CPC G16B 30/00; G16B 20/00; G16B 50/00; G16B 45/00

(57) ABSTRACT

Presented herein are systems and methods related to a scalable and platform-agnostic framework that facilitates the creation of personal genetic profile products. In particular, in certain embodiments, the systems and methods described herein facilitate the creation, storage, organization, and maintenance (e.g. updating) of data corresponding to the relationships between particular variations that may occur in an individual's DNA (e.g. variants of different specific SNPs) and the health-related phenotypes that they influence. The data stored in this manner can be used as a template not only for presenting personal genetic profile assessments to an individual in an informative and user-friendly manner, but also for the rapid, automated creation of personal genetic profile assessments of a plurality of individuals from data corresponding to genotyping measurements of biological samples provided by the individuals.

### CROSS REFERENCE TO RELATED APPLICATIONS

This application claims the benefit of U.S. Provisional Application No. 62/436,947, filed Dec. 20, 2016, U.S. Non-Provisional application Ser. No. 15/445,752, filed Feb. 28, 2017, and U.S. Provisional Application No. 62/485,322, filed Apr. 13, 2017, the contents of each of which are hereby incorporated by reference herein in their entirety.

### FIELD

This invention relates generally to systems and methods for facilitating creation of personal genetic profile products and assessments.

### BACKGROUND

Genomes hold much valuable information that can be used to better understand biological characteristics and traits of humans and animals. Personal genetic profiles can be used by individuals to learn about their genome or genomes of related persons or animals. Biological characteristics and traits influenced or determined by an individual's genome include from easily observable traits such as eye color and height as well as harder to observe or quantify traits and characteristics such as food sensitivities and intellectual and physical aptitudes. Developing an understanding of a genome can provide valuable information that may be used in making decisions about one's behavior and habits.

Until recently, characterizing a genome was prohibitively expensive such that very few individual genomes had been fully or partially characterized. Techniques utilized in genotyping a genome required significant resources that limited genotyping to laboratory use in scientific research and related areas. Developments of cost-effective equipment and procedures for genotyping have made personal genotyping feasible. The output of genetic information from such genotyping procedures still requires expertise in the biological sciences to understand.

At present, much research is being conducted to establish relationships between the human genome and biological characteristics and traits, in particular. Single nucleotide polymorphisms (SNPs) are specific sites identified in particular genes that influence biological characteristics and traits different depending on the particular polymorphism of an individual. Different polymorphisms of the nucleotides at a specific site influence the relevant characteristic or trait differently. The influence of an individual's polymorphism on his/her trait can be positive or negative. Many relationships between the variants of SNPs and their corresponding biological characteristics and traits have been established and many more possible relationships are currently undiscovered and under investigation.

Data from genotyping tests may lack organization and may be difficult to conveniently and meaningfully assemble for presentation to users, particularly where data from large numbers of users are involved. Accordingly, there is a need for systems and methods that facilitate the creation and presentation of personal genetic profile assessments that not only provide an individual with their particular genetic profile assessment, but also convey information related to the particular traits and characteristics that are influenced by the specific SNP variants present in their genetic material. Furthermore, there is a need for systems that streamline the creation of user-friendly products that present such genetic profile assessments.

### SUMMARY OF THE INVENTION

Presented herein are systems and methods related to a scalable and platform-agnostic framework that facilitates the creation of personal genetic profile products. In particular, in certain embodiments, the systems and methods described herein facilitate the creation, storage, organization, and maintenance (e.g. updating) of data corresponding to the relationships between particular variations that may occur in an individual's DNA (e.g. variants of different specific SNPs) and the health-related phenotypes that they influence. The data stored in this manner can be used as a template not only for presenting personal genetic profile assessments to an individual in an informative and user-friendly manner, but also for the rapid, automated creation of personal genetic profile assessments of a plurality of individuals from data corresponding to genotyping measurements of biological samples provided by the individuals.

In certain embodiments, the systems and methods described herein provide for creation, storage, and updating of a first class of data structures, referred to herein as products, which are used to represent different general classes of health-related traits and characteristics. For example, different particular products are used to represent different classes of traits such as those corresponding to (i) the way in which an individual's body processes different foods and nutrients, (ii) skin health, and (iii) physical fitness.

In certain embodiments, each product is associated with one or more of a second class of data structures, referred to as categories. In certain embodiments, each category corresponds to a particular health-related trait or characteristic (e.g. food sensitivity, food breakdown, hunger and weight, vitamins, skin uv sensitivity, endurance, metabolism, joint health, muscle strength, intelligence). In certain embodiments, the categories with which a particular product is associated each correspond to different health-related traits or characteristics that are related to the general class of health-related traits or characteristics to which the particular product corresponds (e.g. the general class of health-related traits or characteristics that the product represents).

In turn, each category is associated with one or more SNP objects and/or gene objects, wherein each SNP object is a data structure that represents a specific, physical SNP, and each gene object is a data structure that represents a specific gene. SNP objects and gene objects are associated with a particular category based on the specific health-related phenotypes the SNPs and genes to which they correspond influence. In particular, SNP objects and gene objects corresponding to SNPs and genes, respectively, that influence specific health related phenotypes related to the trait or characteristic to which a particular category corresponds are associated with that particular category.

In certain embodiments, the systems and methods described herein provide a convenient user interface (e.g. a graphical user interface (GUI)) for creation and/or updating of products, categories, SNP objects, and gene objects, as well as the respective associations between them. The data stored within the framework described herein can thus be updated in a flexible manner in order to account for changes in the body of scientific knowledge regarding the relationships between an individual's genomic profile and their overall health, as well as changes in the desires of individuals to learn about different aspects of their personal genetic profile.

In certain embodiments, the hierarchical organization of product, category, SNP object and gene object data structures serves as a flexible template that facilitates both the rapid creation of individual personal genetic profile assessments from genotyping measurements taken from a plurality of individuals, as well as the presentation of an individual's personal genetic profile assessment. In particular, an individual may purchase assessments corresponding to different products, in order to gain insight into the manner in which their personal genome influences the different general classes of health-related traits and characteristics to which each different product corresponds. Accordingly, an individual's personal genetic profile assessment corresponding to one or more products comprises, for each specific SNP associated with each category that is associated with each of the one or more products, an identification of the particular variant of the specific SNP that the individual has. Typically, the identification is obtained via one or more genotyping measurements performed on a biological sample taken from the individual (e.g. a blood sample, e.g. a cheek swab sample, e.g. a saliva sample).

In certain embodiments, an individual may purchase a first assessment corresponding to a first product, and provide a biological sample for genotyping. The individual's biological sample may be stored (e.g. cryogenically frozen). After a period of time, the individual may choose to purchase additional assessments corresponding to other products, and the individual's previously stored biological sample may be taken from storage for additional genotyping measurements of the additional SNPs that are associated with the new products. Moreover, in certain embodiments, additional new products may be created over time, and new assessments corresponding to new products offered to and purchased by individuals. In certain embodiments, as new information related to the influence of new and/or existing SNPs on different specific health related phenotypes is elucidated, new SNP objects and gene objects may be created, and new associations between them and new or existing categories and/or products established. In certain embodiments, existing personal genetic profile assessments of individuals are automatically updated to reflect such new information.

Thus, by providing a framework comprising a hierarchical organization of data structures corresponding to products, categories, SNP objects, and gene objects, the systems and methods described herein provide an intuitive and flexible approach to storing, updating, and creating new associations between different classes of health-related traits and characteristics, and the underlying genetic variations corresponding to different specific SNPs that influence them.

In one aspect, the invention is directed to a method for creating personal genetic profile products that correspond to an assessment of an individual's genetic profile via a graphical user interface (GUI), the method comprising: (a) presenting, by a processor of a computing device, a graphical user interface element (e.g., widget) for creation of a gene object corresponding to a specific gene (e.g., of the human genome), wherein the gene object is a data structure comprising: one or more SNP objects, each SNP object corresponding to a specific SNP occurring in or nearby (e.g. in a promotor region that influences transcription of the specific gene, e.g. occurring within 5 kb upstream or downstream of the specific gene, e.g. occurring within 100 kb upstream or downstream of the specific gene, e.g. occurring within 500 kb upstream or downstream of the specific gene, e.g. occurring within 1 Mb upstream or downstream of the specific gene) the specific gene with which the gene object is associated, and each SNP object comprising: (i) a SNP reference (e.g. a number or code that identifies the specific SNP corresponding with the SNP object); and (ii) for each of one or more variants of the specific SNP to which the SNP object corresponds: a measurement outcome associated with the variant; a qualifier associated with the variant; and optionally, additional information associated with the variant; and (iii) optionally, additional information associated with the SNP object; and (b) receiving, by the processor, via the graphical user interface element, a SNP object associated with the gene object; (c) associating, by the processor, the received SNP object with the gene object; and (d) storing, by the processor, the gene object for further retrieval and/or updating by the processor via the GUI.

In certain embodiments, the gene object comprises a gene identifier (e.g. an alphanumeric code, e.g. an accepted name of the gene) that identifies the specific gene (e.g. of the human genome) with which the gene object is associated.

In certain embodiments, the SNP reference is an alphanumeric code (e.g. a National Center for Biotechnology Information (NCBI) database reference number) that serves as a unique identifier of the specific SNP with which the SNP object is associated.

In certain embodiments, for each variant of the specific SNP to which the SNP object corresponds, the measurement outcome associated with the variant corresponds to an identification of a result of a genotyping measurement of the specific SNP with which the SNP object that comprises the variant is associated. In certain embodiments, the measurement outcome associated with the variant comprises an identification of a first nucleotide associated with a first copy (e.g. from a first of two sets of chromosomes) of an individual's genetic material, and an identification of a second nucleotide of a second copy (e.g. from a second of two sets of chromosomes) of an individual's genetic material (e.g. the measurement outcome comprises a two letter sequence, each letter identifying a particular nucleotide, e.g. the letter ‘A’ identifies adenine, the letter ‘G’ identifies guanine, e.g. the letter ‘T’ identifies thymine, e.g. the letter ‘C’ identifies cytosine).

In certain embodiments, the SNP object comprises, for each of three physically viable variants of the specific SNP to which the SNP object corresponds: a measurement outcome associated with the variant; and a qualifier associated with the variant. In certain embodiments, for each variant of the specific SNP to which the SNP object corresponds, the qualifier associated with the variant corresponds to at least one of (i) and (ii) as follows: (i) a classification of the variant based on prevalence of the variant within a population; and (ii) a classification of the variant based on a health-related phenotype associated with the variant. In certain embodiments, for each variant of the specific SNP to which the SNP object corresponds, the particular qualifier associated with the variant is a specific qualifier of a countable set (e.g. finite number) of predefined qualifiers (e.g. one of three predefined qualifiers, e.g. selected from the group consisting of “Adapt”, “Normal”, “Gifted”).

In certain embodiments, the SNP object comprises additional information associated with the SNP object, the additional information comprising at least one of: (i) a short description (e.g. 2 word or less, 3 word or less) of the specific SNP to which the SNP object corresponds (e.g. a short characterization of a health-related trait that the specific SNP influences, e.g. “Alcohol Tolerance”, e.g. “Caffeine Metabolism”, e.g. “Lactose Intolerance”, e.g. “Blood Flow Regulation”); (ii) a description (e.g. a paragraph describing results of published studies directed to health-related traits influenced by the specific SNP to which the SNP object corresponds); and (iii) one or more references, each reference identifying a source of information (e.g. a citation identifying a published study, e.g. a weblink).

In certain embodiments, the SNP object comprises, for each variant of the specific SNP to which the SNP object corresponds, additional information associated with the variant, the additional information comprising a description of the variant (e.g. a description of a specific health-related phenotype that an individual with the particular variant exhibits).

In certain embodiments, the method comprises receiving a user input to initiate creation of the gene object (e.g. comprising entering a name of the gene object into the GUI, e.g. clicking a button).

In certain embodiments, receiving the SNP object associated with the gene object comprises receiving, by the processor, via the graphical user interface element, a user input of: (i) a SNP reference; and (ii) for each of one or more variants, a measurement outcome associated with the variant and a qualifier associated with the variant.

In certain embodiments, step (a) of the method comprises: presenting a SNP reference graphical control element for user entry of a SNP reference (e.g. a text box); and for each of one or more variants to be represented in (e.g., added to) a SNP object, presenting: (i) a respective variant measurement graphical control element (e.g. a text box) for user entry of a measurement outcome associated with the respective variant; and (ii) a respective variant qualifier graphical control element (e.g. a drop down list that displays a set of one or more predefined qualifiers) for user selection of a qualifier associated with the respective variant, and step (b) of the method comprises: receiving, via the SNP reference graphical control element, a user input of a SNP reference; and for each of the one or more variants to be represented in (e.g., added to) the SNP object: (i) receiving, via the respective variant measurement graphical control element, a user input of a measurement outcome; and (ii) receiving, via the respective variant qualifier graphical control element, a user selection of a qualifier.

In certain embodiments, the method comprises: (e) presenting, by the processor, a graphical user interface element for creation of a category corresponding to a health-related phenotype (e.g. skin uv sensitivity, e.g. endurance, e.g. metabolism, e.g. joint health, e.g. muscle strength, e.g. food sensitivity, e.g. vitamin levels, e.g. intelligence) associated with a predefined group of one or more related SNPs each of which is associated with the health-related phenotype, wherein the category is a data structure comprising one or more SNP objects; (f) receiving, by the processor, via the graphical user interface element for creation of a category, a selection of one or more SNP objects; (g) associating, by the processor, the one or more selected SNP objects with the category; and (h) storing, by the processor, the category, for further retrieval and/or updating by the processor via the GUI.

In certain embodiments, the category comprises additional information selected from the group consisting of: (i) a name of the category; (ii) a background image associated with the category; (iii) an icon associated with the category; (iv) a category order identifier associated with the category; and (v) a description of the category.

In certain embodiments, step (e) comprises presenting (i) a first gene selection graphical control element (e.g. a drop down list) for selecting a first gene object and (ii) a first SNP selection graphical control element (e.g. a drop down list) for selecting a first SNP object of the first gene object, and step (f) comprises receiving, via the first gene selection graphical control element, a user selection of a first gene object, and receiving, via the first SNP selection graphical control element, a user selection of a first SNP object of the selected first gene object.

In certain embodiments, the first gene selection graphical control element displays a list of selectable elements, each element of the list corresponding to a previously stored gene object, and following a user selection of the first gene object, the first SNP selection graphical control element displays a list of selectable elements, each element of the list corresponding to a SNP object of the previously selected first gene object.

In certain embodiments, the method comprises: receiving, by the processor, a user input to add more genes to the category; and responsive to the user input to add more genes to the category, presenting (i) a second gene selection graphical control element for selecting a second gene object and (ii) a second SNP selection graphical control element for selecting a second SNP object of the second gene object.

In certain embodiments, the method comprises: (i) presenting, by the processor, a graphical user interface element for creation of a product corresponding to a general class of health-related phenotypes (e.g. overall skin health (e.g. AURA™), e.g. fitness (e.g. FITCODE™), e.g. nutritional health (e.g. FUEL™), e.g. exceptional qualities (e.g. SUPERHERO™) associated with a predefined group of one or more related categories, wherein the product is a data structure comprising one or more categories; (j) receiving, by the processor, via the graphical user interface element for creation of the product, a selection of one or more categories; (k) associating, by the processor, the one or more selected categories with the product; and (l) storing, by the processor, the product for further retrieval and/or updating by the GUI.

In certain embodiments, the product comprises additional information selected from the group consisting of: (i) a name of the product; (ii) an icon associated with the product; and (iii) a description of the product.

In another aspect, the invention is directed to method of creating and/or updating personal genetic profile assessments for a plurality of individuals, the method comprising: receiving, by a processor of a computing device, genotyping data collected from a plurality of biological samples, each biological sample having been taken from a particular individual of the plurality of individuals, wherein the genotyping data comprises, for each biological sample, one or more genotyping measurements of one or more SNPs, each SNP associated with one or more genes (e.g. each SNP occurring within one of the one or more genes or occurring nearby one or more genes (e.g. within a promotor region that influences transcription of one or more genes, e.g. within 5 kb upstream or downstream of one or more genes, e.g. within 100 kb upstream or downstream of one or more genes, e.g. occurring within 500 kb upstream or downstream of one or more genes, e.g. within 1 Mb upstream or downstream of one or more genes)); and for each individual associated with each biological sample, for each SNP (e.g. for each SNP that was measured for the associated biological sample): (a) identifying, by the processor, a gene object (e.g. a gene object created via any of the methods described above) corresponding to a target gene of the one or more genes with which the SNP is associated and a SNP object (e.g. a SNP object as created via any of the methods described above) of the gene object corresponding to the SNP; (b) receiving, by the processor, a determination of a particular variant of the SNP that the individual has and determining a qualifier (e.g. a qualifier as described in any of the embodiments described above) associated with the particular variant; and (c) storing, by the processor, within the personal genetic profile assessment of the individual: (i) a gene identifier (e.g. a gene identifier of the identified gene object) associated with the measured SNP, wherein the gene identifier identifies the target gene; (ii) a SNP reference (e.g. the SNP reference of the SNP object as created via any of the methods described above) that identifies the measured SNP, wherein the SNP reference is associated with the gene identifier; (iii) a measurement outcome that identifies the particular variant of the SNP that the individual has, wherein the measurement outcome is associated with the SNP reference; and (iv) the qualifier (e.g. a qualifier as described in any of the embodiments described above) associated with the particular variant, wherein the qualifier is associated with the SNP reference.

In certain embodiments, for each biological sample, the one or more genotyping measurements of the one or more SNPs comprise(s), for each SNP, a first measurement that identifies a first nucleotide of a first copy (e.g. from a first of two sets of chromosomes) of an individual's genetic material, and a second measurement that identifies a second nucleotide of a second copy (e.g. from a second of two sets of chromosomes) of an individual's genetic material. In certain embodiments, the genotyping data comprises data from a PCR-based SNP genotyping assay.

In certain embodiments, the method comprises measuring, via a PCR-based SNP genotyping assay, one or more SNPs for a plurality of biological samples, thereby producing genotyping data.

In certain embodiments, for each biological sample: the genotyping data comprises, for each gene with which each SNP is associated, a corresponding gene identifier, and the genotyping data comprises, for each SNP, a corresponding SNP reference, and step (a) comprises, for each measured SNP: accessing, by the processor, a plurality of previously stored gene objects (e.g. gene objects created and stored via any of the methods described above), wherein each previously stored gene object is a data structure comprising a gene identifier and one or more SNP objects, each SNP object of a previously stored gene object comprising a SNP reference; matching, by the processor, a gene identifier (e.g. of one of the one or more genes with which the measured SNP is associated) of the genotyping data to a gene identifier (e.g. a gene identifier as described above) of one of the plurality of previously stored gene objects, thereby identifying the gene object associated with the gene to which the SNP belongs; and matching, by the processor, the SNP reference (corresponding to the measured SNP) of the genotyping data to a SNP reference of one of the SNP objects (e.g. the SNP reference of the SNP objects created via any of the methods described above) of the plurality of previously stored gene objects, thereby identifying the SNP object of the gene object corresponding to the SNP, thereby identifying the gene object corresponding to the target gene with which the SNP is associated and the SNP object corresponding to the SNP. In certain embodiments, the genotyping data comprises, for each SNP, a corresponding SNP reference, and for each biological sample, for each measured SNP, determining, by the processor, a particular variant of the SNP that the individual associated with the biological sample has and a qualifier associated with the variant comprises: accessing, by the processor, a plurality of previously stored gene objects (e.g. gene objects previously created and stored via any of the methods described above), wherein each gene object comprises: one or more SNP objects, each SNP object comprising: a SNP reference (e.g. a number or code that identifies a specific SNP associated with the SNP object); and for each of one or more variants of a specific SNP associated with the SNP object, a measurement outcome associated with the variant, and a qualifier associated with the variant; matching, by the processor, the SNP reference (corresponding to the measured SNP) of the genotyping data to a SNP reference of one of the SNP objects of the plurality of previously stored gene objects, thereby identifying a SNP object corresponding to the measured SNP; and matching, by the processor, the genotyping measurement of the measured SNP to a measurement outcome of identified SNP object corresponding to the measured SNP, thereby determining the particular variant of the SNP that the individual has.

In certain embodiments, the method comprises automatically determining, by the processor, for each individual of the plurality of individuals associated with the plurality of measured biological samples, one or more products (e.g. products created via any of the methods described above) associated with the individual's personal genetic profile assessment, wherein: each product corresponds to a predefined group of one or more related categories (e.g. the categories as created via any of the methods described above), and the product is a data structure comprising one or more categories, each category corresponds to a predefined group of one or more related SNPs, each of which is associated with a health-related phenotype, wherein the category is a data structure comprising one or more SNP objects (e.g. each SNP object belonging to a gene object previously created and stored via any of the methods described above), and for each individual of the plurality of individuals, automatically determining one or more products associated with the individual's personal genetic profile assessment comprises, for each product of a plurality of previously stored products: determining a list of all the different SNP objects that the product comprises, the list comprising each distinct SNP object of each category the product comprises; and matching, by the processor, the list of all the different SNP objects of the product to the measured SNPs of the genotyping data collected from the biological sample associated with the individual.

In certain embodiments, the method comprises, for an individual of the plurality of individuals, causing, by the processor, display (e.g. on a computing device associated with the individual) of an assessment graphical user interface (GUI) for view of the individual's personal genetic profile assessment, the assessment GUI comprising, for each measured SNP associated with the individual, a graphical element comprising: (A) at least one of (i) or (ii) as follows: (i) a graphical representation of the associated (with the measured SNP) gene identifier of the individual's personal genetic profile assessment; (ii) a graphical representation of the SNP reference of the individual's personal genetic profile assessment that identifies the measured SNP; and (B) a graphical representation of the qualifier of the individual's personal genetic profile assessment, wherein the qualifier is the qualifier that is associated with the SNP reference that identifies the measured SNP.

In certain embodiments, the method comprises: causing, by the processor, display (e.g. on a computing device associated with the individual) of one or more selectable product graphical control elements, each selectable product graphical control element corresponding to a previously stored product, wherein: each previously stored product corresponds to a predefined group of one or more related categories, and each category corresponds to a predefined group of one or more related gene-SNP combinations each of which is associated with the health-related phenotype; responsive to a user selection of a specific product graphical control element, for each category of the previously stored product corresponding to the selected product graphical control element, causing display of a respective selectable category graphical control element; and responsive to a user selection of a specific category graphical control element, for each gene-SNP combination of the corresponding category, causing display of a corresponding gene graphical control element, wherein each gene graphical control element comprises: at least one of (i) or (ii) as follows: (i) a graphical representation of the associated (with the measured SNP) gene identifier of the individual's personal genetic profile assessment; (ii) a graphical representation of the SNP reference of the individual's personal genetic profile assessment that identifiers the measured SNP; and a graphical representation of the qualifier of the individual's personal genetic profile assessment, wherein the qualifier is the qualifier that is associated with the SNP reference that identifies the measured SNP.

In certain embodiments, the method comprises: receiving, by the processor, a command to share data (e.g., due to selection, by the individual, of a graphical control element for sharing) corresponding to at least a portion of the personal genetic profile assessment of an individual of the plurality of individuals; receiving, by the processor, an identification of a sharing entity [e.g., an email address, e.g., a phone number, e.g., an address of a web service (e.g., a social media website), e.g., a member identifier associated with an individual of the plurality of individuals] with which to share the data corresponding to the genetic profile assessment of the individual; and responsive to receipt of the command to share data and the identification sharing entity, providing, by the processor, the data corresponding to the at least a portion of the personal genetic profile assessment of the individual to the sharing entity (e.g., via a generated report; e.g., via a link to a website; e.g., providing data to a GUI on a computing device of the sharing entity). In certain embodiments, the command to share data corresponding to at least a portion of the personal genetic profile assessment of the individual comprises an identification of a shareable portion (up to all) of the measured SNPs associated with the individual (e.g., a list of specific measured SNPs; e.g., an identification of one or more categories of products) and the data provided to the sharing entity includes only data corresponding each measured SNP of the shareable portion of the measured SNPs associated with the individual. In certain embodiments, the data corresponding to the at least a portion of the individual's personal genetic profile assessment comprises, for each measured SNP of a shareable portion (up to all) of the measured SNPs associated with the individual: at least one of: (i) the gene identifier associated with the measured SNP; and (ii) the SNP reference associated with the measured SNP; and at least one of: (iii) the particular variant of the SNP that the individual has; and (iv) the qualifier associated with the particular variant of the SNP that the individual has. In certain embodiments, providing the data corresponding to the portion of the personal genetic profile assessment of the individual to the sharing entity comprises: generating an assessment report (e.g., a shareable document) comprising, for each measured SNP of a portion (up to all) of the measured SNPs associated with the individual: a first graphical representation corresponding to a graphical representation of at least one of: (i) the gene identifier associated with the measured SNP; and (ii) the SNP reference associated with the measured SNP; and in relation (e.g., in spatial proximity to, such as in a same row of a table) to the first graphical representation, a second graphical representation corresponding to a graphical representation of at least one of: (iii) the particular variant of the SNP that the individual has; and (iv) the qualifier associated with the particular variant of the SNP that the individual has; and providing, by the processor, the assessment report to the sharing entity (e.g., sending via email, text message, a web link from which the assessment report can be downloaded). In certain embodiments, providing the data corresponding to the portion of the personal genetic profile assessment of the individual to the sharing entity comprises causing, by the processor, display (e.g. on a computing device associated with the sharing entity) of the assessment graphical user interface (GUI) described herein.

In certain embodiments, the data corresponding to the at least a portion of the personal genetic profile assessment of the individual comprises an identification a shareable portion (up to all) of the measured SNPs associated with the individual, each measured SNP of the shared portion corresponding to a shareable SNP, and providing the data corresponding to the portion of the personal genetic profile assessment of the individual to the sharing entity comprises causing, by the processor, display (e.g. on a computing device associated with the sharing entity) of an assessment graphical user interface (GUI) for view of the portion of the personal genetic profile assessment of the individual, the assessment GUI comprising, for each shareable SNP, a graphical element comprising: (A) at least one of (i) or (ii) as follows: (i) a graphical representation of the associated (with the shareable SNP) gene identifier of the personal genetic profile assessment of the individual; and (ii) a graphical representation of the SNP reference of the personal genetic profile assessment of the individual, wherein the SNP reference identifies the shareable SNP; and (B) a graphical representation of the qualifier of the personal genetic profile assessment individual, wherein the qualifier is the qualifier that is associated with the SNP reference that identifies the shareable SNP.

In certain embodiments, the causing display of the assessment GUI for view of the at least a portion of the personal genetic profile assessment of the individual comprises: causing, by the processor, display (e.g. on a computing device associated with the individual) of one or more selectable product graphical control elements, each selectable product graphical control element corresponding to a previously stored product, wherein: each previously stored product corresponds to a predefined group of one or more related categories, and each category corresponds to a predefined group of one or more related gene-SNP combinations each of which is associated with the health-related phenotype; responsive to a user selection of a specific product graphical control element, for each category of the previously stored product corresponding to the selected product graphical control element, causing display of a respective selectable category graphical control element; and responsive to a user selection of a specific category graphical control element, for each gene-SNP combination of the corresponding category and corresponding to a shareable SNP, causing display of a corresponding gene graphical control element, wherein each gene graphical control element comprises: at least one of (i) or (ii) as follows: (i) a graphical representation of the associated (with the shareable SNP) gene identifier of the individual's personal genetic profile assessment; and (ii) a graphical representation of the SNP reference of the individual's personal genetic profile assessment that identifiers the shareable SNP; and a graphical representation of the qualifier of the individual's personal genetic profile assessment, wherein the qualifier is the qualifier that is associated with the SNP reference that identifies the shareable SNP.

In another aspect, the invention is directed to a system of creating and/or updating personal genetic profile assessments for a plurality of individuals, the system comprising: a processor; a non-transitory computer readable memory having instructions stored thereon, wherein the instructions, when executed by the processor, cause the processor to perform any of the methods described herein.

In another aspect, the invention is directed to a system for creating personal genetic profile products that correspond to an assessment of an individual's genetic profile via a graphical user interface (GUI), the system comprising: a processor; and a memory having instructions stored thereon, wherein the instructions, when executed by the processor, cause the processor to: (a) present a graphical user interface element (e.g., widget) for creation of a gene object corresponding to a specific gene (e.g., of the human genome), wherein the gene object is a data structure comprising: one or more SNP objects, each SNP object corresponding to a specific SNP occurring in or nearby (e.g. in a promotor region that influences transcription of the specific gene, e.g. occurring within 5 kb upstream or downstream of the specific gene, e.g. occurring within 100 kb upstream or downstream of the specific gene, e.g. occurring within 500 kb upstream or downstream of the specific gene, e.g. occurring within 1 Mb upstream or downstream of the specific gene) the specific gene with which the gene object is associated, and each SNP object comprising: (i) a SNP reference (e.g. a number or code that identifies the specific SNP corresponding with the SNP object); and (ii) for each of one or more variants of the specific SNP to which the SNP object corresponds: a measurement outcome associated with the variant; a qualifier associated with the variant; and optionally, additional information associated with the variant; and (iii) optionally, additional information associated with the SNP object; and (b) receive, via the graphical user interface element, a SNP object associated with the gene object; (c) associate the received SNP object with the gene object; and (d) store the gene object for further retrieval and/or updating by the processor via the GUI.

In certain embodiments, the gene object comprises a gene identifier (e.g. an alphanumeric code, e.g. an accepted name of the gene) that identifies the specific gene (e.g. of the human genome) with which the gene object is associated.

In certain embodiments, the SNP reference is an alphanumeric code (e.g. a National Center for Biotechnology Information (NCBI) database reference number) that serves as a unique identifier of the specific SNP with which the SNP object is associated.

In certain embodiments, for each variant of the specific SNP to which the SNP object corresponds, the measurement outcome associated with the variant corresponds to an identification of a result of a genotyping measurement of the specific SNP with which the SNP object that comprises the variant is associated. In certain embodiments, the measurement outcome associated with the variant comprises an identification of a first nucleotide associated with a first copy (e.g. from a first of two sets of chromosomes) of an individual's genetic material, and an identification of a second nucleotide of a second copy (e.g. from a second of two sets of chromosomes) of an individual's genetic material (e.g. the measurement outcome comprises a two letter sequence, each letter identifying a particular nucleotide, e.g. the letter ‘A’ identifies adenine, the letter ‘G’ identifies guanine, e.g. the letter ‘T’ identifies thymine, e.g. the letter ‘C’ identifies cytosine).

In certain embodiments, the SNP object comprises, for each of three physically viable variants of the specific SNP to which the SNP object corresponds: a measurement outcome associated with the variant; and a qualifier associated with the variant. In certain embodiments, for each variant of the specific SNP to which the SNP object corresponds, the qualifier associated with the variant corresponds to at least one of (i) and (ii) as follows: (i) a classification of the variant based on prevalence of the variant within a population; and (ii) a classification of the variant based on a health-related phenotype associated with the variant. In certain embodiments, for each variant of the specific SNP to which the SNP object corresponds, the particular qualifier associated with the variant is a specific qualifier of a countable set (e.g. finite number) of predefined qualifiers (e.g. one of three predefined qualifiers, e.g. selected from the group consisting of “Adapt”, “Normal”, “Gifted”).

In certain embodiments, the SNP object comprises additional information associated with the SNP object, the additional information comprising at least one of: (i) a short description (e.g. 2 word or less, 3 word or less) of the specific SNP to which the SNP object corresponds (e.g. a short characterization of a health-related trait that the specific SNP influences, e.g. “Alcohol Tolerance”, e.g. “Caffeine Metabolism”, e.g. “Lactose Intolerance”, e.g. “Blood Flow Regulation”); (ii) a description (e.g. a paragraph describing results of published studies directed to health-related traits influenced by the specific SNP to which the SNP object corresponds); and (iii) one or more references, each reference identifying a source of information (e.g. a citation identifying a published study, e.g. a weblink).

In certain embodiments, the SNP object comprises, for each variant of the specific SNP to which the SNP object corresponds, additional information associated with the variant, the additional information comprising a description of the variant (e.g. a description of a specific health-related phenotype that an individual with the particular variant exhibits).

In certain embodiments, the instructions cause the processor to receive a user input to initiate creation of the gene object (e.g. comprising entering a name of the gene object into the GUI, e.g. clicking a button).

In certain embodiments, the instructions cause the processor to receive the SNP object associated with the gene object by receiving, via the graphical user interface element, a user input of: (i) a SNP reference; and (ii) for each of one or more variants, a measurement outcome associated with the variant and a qualifier associated with the variant.

In certain embodiments, the instructions cause the processor to: at step (a): present a SNP reference graphical control element for user entry of a SNP reference (e.g. a text box); and for each of one or more variants to be represented in (e.g., added to) a SNP object, present: (i) a respective variant measurement graphical control element (e.g. a text box) for user entry of a measurement outcome associated with the respective variant; and (ii) a respective variant qualifier graphical control element (e.g. a drop down list that displays a set of one or more predefined qualifiers) for user selection of a qualifier associated with the respective variant; and at step (b): receive, via the SNP reference graphical control element, a user input of a SNP reference; and for each of the one or more variants to be represented in (e.g., added to) the SNP object: (i) receive, via the respective variant measurement graphical control element, a user input of a measurement outcome; and (ii) receive, via the respective variant qualifier graphical control element, a user selection of a qualifier.

In certain embodiments, the instructions cause the processor to: (e) present a graphical user interface element for creation of a category corresponding to a health-related phenotype (e.g. skin uv sensitivity, e.g. endurance, e.g. metabolism, e.g. joint health, e.g. muscle strength, e.g. food sensitivity, e.g. vitamin levels, e.g. intelligence) associated with a predefined group of one or more related SNPs each of which is associated with the health-related phenotype, wherein the category is a data structure comprising one or more SNP objects; (f) receive, via the graphical user interface element for creation of a category, a selection of one or more SNP objects; (g) associate the one or more selected SNP objects with the category; and (h) store the category for further retrieval and/or updating by the processor via the GUI.

In certain embodiments, the category comprises additional information selected from the group consisting of: (i) a name of the category; (ii) a background image associated with the category; (iii) an icon associated with the category; (iv) a category order identifier associated with the category; and (v) a description of the category.

In certain embodiments, the instructions cause the processor to: at step (e), present (i) a first gene selection graphical control element (e.g. a drop down list) for selecting a first gene object and (ii) a first SNP selection graphical control element (e.g. a drop down list) for selecting a first SNP object of the first gene object; and at step (f): receive, via the first gene selection graphical control element, a user selection of a first gene object; and receive, via the first SNP selection graphical control element, a user selection of a first SNP object of the selected first gene object.

In certain embodiments, the first gene selection graphical control element displays a list of selectable elements, each element of the list corresponding to a previously stored gene object, and following a user selection of the first gene object, the first SNP selection graphical control element displays a list of selectable elements, each element of the list corresponding to a SNP object of the previously selected first gene object.

In certain embodiments, the instructions cause the processor to: receive a user input to add more genes to the category; and responsive to the user input to add more genes to the category, present (i) a second gene selection graphical control element for selecting a second gene object and (ii) a second SNP selection graphical control element for selecting a second SNP object of the second gene object.

In certain embodiments, the instructions cause the processor to: (i) present a graphical user interface element for creation of a product corresponding to a general class of health-related phenotypes (e.g. overall skin health (e.g. AURA™), e.g. fitness (e.g. FITCODE™), e.g. nutritional health (e.g. FUEL™), e.g. exceptional qualities (e.g. SUPERHERO™) associated with a predefined group of one or more related categories, wherein the product is a data structure comprising one or more categories; (j) receive, via the graphical user interface element for creation of the product, a selection of one or more categories; (k) associate the one or more selected categories with the product; and (1) store the product for further retrieval and/or updating by the GUI.

In certain embodiments, the product comprises additional information selected from the group consisting of: (i) a name of the product; (ii) an icon associated with the product; and (iii) a description of the product.

In another aspect, the invention is directed to a system for creating and/or updating personal genetic profile assessments for a plurality of individuals, the system comprising: a processor; and a memory having instructions stored thereon, wherein the instructions, when executed by the processor, cause the processor to: receive genotyping data collected from a plurality of biological samples, each biological sample having been taken from a particular individual of the plurality of individuals, wherein the genotyping data comprises, for each biological sample, one or more genotyping measurements of one or more SNPs, each SNP associated with one or more genes (e.g. each SNP occurring within one of the one or more genes or occurring nearby one or more genes (e.g. within a promotor region that influences transcription of one or more genes, e.g. within 5 kb upstream or downstream of one or more genes, e.g. within 100 kb upstream or downstream of one or more genes, e.g. occurring within 500 kb upstream or downstream of one or more genes, e.g. within 1 Mb upstream or downstream of one or more genes)); and for each individual associated with each biological sample, for each SNP (e.g. for each SNP that was measured for the associated biological sample): (a) identify a gene object (e.g. a gene object created via any of the systems described above) corresponding to a target gene of the one or more genes with which the SNP is associated and a SNP object (e.g. a SNP object of any of the systems described above) of the gene object corresponding to the SNP; (b) receive a determination of a particular variant of the SNP that the individual has and determining a qualifier (e.g. a qualifier as described in any of the embodiments described above) associated with the particular variant; and (c) store within the personal genetic profile assessment of the individual: (i) a gene identifier (e.g. a gene identifier of the identified gene object) associated with the measured SNP, wherein the gene identifier identifies the target gene; (ii) a SNP reference (e.g. the SNP reference of the SNP object of any of the systems described above) that identifies the measured SNP, wherein the SNP reference is associated with the gene identifier; (iii) a measurement outcome that identifies the particular variant of the SNP that the individual has, wherein the measurement outcome is associated with the SNP reference; and (iv) the qualifier (e.g. a qualifier as described in any of the embodiments described above) associated with the particular variant, wherein the qualifier is associated with the SNP reference.

In certain embodiments, for each biological sample, the one or more genotyping measurements of the one or more SNPs comprise(s), for each SNP, a first measurement that identifies a first nucleotide of a first copy (e.g. from a first of two sets of chromosomes) of an individual's genetic material, and a second measurement that identifies a second nucleotide of a second copy (e.g. from a second of two sets of chromosomes) of an individual's genetic material. In certain embodiments, the genotyping data comprises data from a PCR-based SNP genotyping assay.

In certain embodiments, the system comprises a reader (e.g. a PCR system reader for measuring PCR-based genotyping assays) for measuring genotyping data for one or more SNPs for a plurality of biological samples and providing the genotyping data to the processor.

In certain embodiments, for each biological sample: the genotyping data comprises, for each gene with which each SNP is associated, a corresponding gene identifier, and the genotyping data comprises, for each SNP, a corresponding SNP reference, and the instructions cause the processor to, at step (a), for each measured SNP: access a plurality of previously stored gene objects (e.g. gene objects created and stored via any of the systems described above), wherein each previously stored gene object is a data structure comprising a gene identifier and one or more SNP objects, each SNP object of a previously stored gene object comprising a SNP reference; match a gene identifier (e.g. of one of the one or more genes with which the measured SNP is associated) of the genotyping data to a gene identifier (e.g. a gene identifier as described above) of one of the plurality of previously stored gene objects, thereby identifying the gene object associated with the gene to which the SNP belongs; and match the SNP reference (corresponding to the measured SNP) of the genotyping data to a SNP reference of one of the SNP objects (e.g. the SNP reference of the SNP objects created via any of the systems described above) of the plurality of previously stored gene objects, thereby identifying the SNP object of the gene object corresponding to the SNP, thereby identifying the gene object corresponding to the target gene with which the SNP is associated and the SNP object corresponding to the SNP.

In certain embodiments, the genotyping data comprises, for each SNP, a corresponding SNP reference, and the instructions cause the processor to, for each biological sample, for each measured SNP, determine a particular variant of the SNP that the individual associated with the biological sample has and a qualifier associated with the variant by: accessing a plurality of previously stored gene objects (e.g. gene objects previously created and stored via any of the systems described above), wherein each gene object comprises: one or more SNP objects, each SNP object comprising: a SNP reference (e.g. a number or code that identifies a specific SNP associated with the SNP object); and for each of one or more variants of a specific SNP associated with the SNP object, a measurement outcome associated with the variant, and a qualifier associated with the variant; matching the SNP reference (corresponding to the measured SNP) of the genotyping data to a SNP reference of one of the SNP objects of the plurality of previously stored gene objects, thereby identifying a SNP object corresponding to the measured SNP; and matching the genotyping measurement of the measured SNP to a measurement outcome of identified SNP object corresponding to the measured SNP, thereby determining the particular variant of the SNP that the individual has.

In certain embodiments, the instructions cause the processor to, for each individual of the plurality of individuals associated with the plurality of measured biological samples, automatically determine one or more products (e.g. products created via any of the systems described above) associated with the individual's personal genetic profile assessment, wherein: each product corresponds to a predefined group of one or more related categories (e.g. categories created via any of the systems described above), and the product is a data structure comprising one or more categories, each category corresponds to a predefined group of one or more related SNPs, each of which is associated with a health-related phenotype, wherein the category is a data structure comprising one or more SNP objects (e.g. each SNP object belonging to a gene object previously created and stored via any of the systems described above), and for each individual of the plurality of individuals, the instructions cause the processor to determine one or more products associated with the individual's personal genetic profile assessment by, for each product of a plurality of previously stored products: determining a list of all the different SNP objects that the product comprises, the list comprising each distinct SNP object of each category the product comprises; and matching the list of all the different SNP objects of the product to the measured SNPs of the genotyping data collected from the biological sample associated with the individual.

In certain embodiments, the instructions cause the processor to, for an individual of the plurality of individuals, cause display (e.g. on a computing device associated with the individual) of an assessment graphical user interface (GUI) for view of the individual's personal genetic profile assessment, the assessment GUI comprising, for each measured SNP associated with the individual, a graphical element comprising: (A) at least one of (i) or (ii) as follows: (i) a graphical representation of the associated (with the measured SNP) gene identifier of the individual's personal genetic profile assessment; and (ii) a graphical representation of the SNP reference of the individual's personal genetic profile assessment that identifies the measured SNP; and (B) a graphical representation of the qualifier of the individual's personal genetic profile assessment, wherein the qualifier is the qualifier that is associated with the SNP reference that identifies the measured SNP.

In certain embodiments, the instructions cause the processor to: cause display (e.g. on a computing device associated with the individual) of one or more selectable product graphical control elements, each selectable product graphical control element corresponding to a previously stored product, wherein: each previously stored product corresponds to a predefined group of one or more related categories, and each category corresponds to a predefined group of one or more related gene-SNP combinations each of which is associated with the health-related phenotype; responsive to a user selection of a specific product graphical control element, for each category of the previously stored product corresponding to the selected product graphical control element, cause display of a respective selectable category graphical control element; responsive to a user selection of a specific category graphical control element, for each gene-SNP combination of the corresponding category, cause display of a corresponding gene graphical control element, wherein each gene graphical control element comprises: at least one of (i) or (ii) as follows: (i) a graphical representation of the associated (with the measured SNP) gene identifier of the individual's personal genetic profile assessment; and (ii) a graphical representation of the SNP reference of the individual's personal genetic profile assessment that identifiers the measured SNP; and a graphical representation of the qualifier of the individual's personal genetic profile assessment, wherein the qualifier is the qualifier that is associated with the SNP reference that identifies the measured SNP.

In certain embodiments, the instructions cause the processor to: receive a command to share data (e.g., due to selection, by the individual, of a graphical control element for sharing) corresponding to at least a portion of the personal genetic profile assessment of an individual of the plurality of individuals; receive an identification of a sharing entity [e.g., an email address, e.g., a phone number, e.g., an address of a web service (e.g., a social media website), e.g., a member identifier associated with an individual of the plurality of individuals] with which to share the data corresponding to the genetic profile assessment of the individual; and responsive to receipt of the command to share data and the identification sharing entity, provide the data corresponding to the at least a portion of the personal genetic profile assessment of the individual to the sharing entity (e.g., via a generated report; e.g., via a link to a website; e.g., providing data to a GUI on a computing device of the sharing entity).

In certain embodiments, the command to share data corresponding to the at least a portion of the personal genetic profile assessment of the individual comprises an identification of a shareable portion (up to all) of the measured SNPs associated with the individual (e.g., a list of specific measured SNPs; e.g., an identification of one or more categories of products) and the data provided to the sharing entity includes only data corresponding each measured SNP of the identified shareable portion of the measured SNPs associated with the individual.

In certain embodiments, the data corresponding to the at least a portion of the individual's personal genetic profile assessment comprises, for each measured SNP of a shareable portion (up to all) of the measured SNPs associated with the individual: at least one of: (i) the gene identifier associated with the measured SNP; and (ii) the SNP reference associated with the measured SNP; and at least one of: (iii) the particular variant of the SNP that the individual has; and (iv) the qualifier associated with the particular variant of the SNP that the individual has.

In certain embodiments, the instructions cause the processor to provide the data corresponding to the portion of the personal genetic profile assessment of the individual to the sharing entity by: generating an assessment report (e.g., a shareable document) comprising, for each measured SNP of a shareable portion (up to all) of the measured SNPs associated with the individual: a first graphical representation corresponding to a graphical representation of at least one of: (i) the gene identifier associated with the measured SNP; and (ii) the SNP reference associated with the measured SNP; and in relation (e.g., in spatial proximity to, such as in a same row of a table) to the first graphical representation, a second graphical representation corresponding to a graphical representation of at least one of: (iii) the particular variant of the SNP that the individual has; and (iv) the qualifier associated with the particular variant of the SNP that the individual has; and providing the assessment report to the sharing entity (e.g., sending via email, text message, a web link from which the assessment report can be downloaded).

In certain embodiments the instructions cause the processor to provide the data corresponding to the at least a portion of the personal genetic profile assessment of the individual to the sharing entity by causing display (e.g., on a computing device associated with the sharing entity) of the assessment graphical user interface (GUI) described herein.

In certain embodiments, the data corresponding to the at least a portion of the personal genetic profile assessment of the individual comprises an identification a shareable portion (up to all) of the measured SNPs associated with the individual, each measured SNP of the shareable portion corresponding to a shareable SNP, and wherein the instructions cause the processor to provide the data corresponding to the portion of the personal genetic profile assessment of the individual to the sharing entity by causing display (e.g. on a computing device associated with the sharing entity) of an assessment graphical user interface (GUI) for view of the portion of the personal genetic profile assessment of the individual, the assessment GUI comprising, for each shareable SNP, a graphical element comprising: (A) at least one of (i) or (ii) as follows: (i) a graphical representation of the associated (with the shareable SNP) gene identifier of the personal genetic profile assessment of the individual; (ii) a graphical representation of the SNP reference of the personal genetic profile assessment of the individual, wherein the SNP reference identifies the shareable SNP; and (B) a graphical representation of the qualifier of the personal genetic profile assessment individual, wherein the qualifier is the qualifier that is associated with the SNP reference that identifies the shareable SNP.

In certain embodiments, the instructions cause the processor to: cause display (e.g. on a computing device associated with the individual) of one or more selectable product graphical control elements, each selectable product graphical control element corresponding to a previously stored product, wherein: each previously stored product corresponds to a predefined group of one or more related categories, and each category corresponds to a predefined group of one or more related gene-SNP combinations each of which is associated with the health-related phenotype; responsive to a user selection of a specific product graphical control element, for each category of the previously stored product corresponding to the selected product graphical control element, cause display of a respective selectable category graphical control element; and responsive to a user selection of a specific category graphical control element, for each gene-SNP combination of the corresponding category and corresponding to a shareable SNP, cause display of a corresponding gene graphical control element, wherein each gene graphical control element comprises: at least one of (i) or (ii) as follows: (i) a graphical representation of the associated (with the shareable SNP) gene identifier of the individual's personal genetic profile assessment; and (ii) a graphical representation of the SNP reference of the individual's personal genetic profile assessment that identifiers the shareable SNP; and a graphical representation of the qualifier of the individual's personal genetic profile assessment, wherein the qualifier is the qualifier that is associated with the SNP reference that identifies the shareable SNP.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 is a block diagram illustrating associations between different data structures provided in accordance with the systems and methods described herein, according to an illustrative embodiment;

FIG. 2 is a block diagram showing an organizational hierarchy of a personal genetic profile product, according to an illustrative embodiment;

FIG. 3A is a screenshot showing a homescreen of a graphical user interface (GUI) that a user uses to view different products that summarize their genetic profile, according to an illustrative embodiment;

FIG. 3B is a screenshot of the GUI of FIG. 3A showing the interface that appears when a particular product is selected, according to an illustrative embodiment;

FIG. 3C is a screenshot of the GUI showing a summary of the product that appears when the information (“i”) button of FIG. 3B is selected, according to an illustrative embodiment;

FIG. 3D is a screenshot of the GUI of FIG. 3A showing the interface that appears when a particular category of the selected product is selected, according to an illustrative embodiment;

FIG. 3E is a screenshot of the GUI of FIG. 3A showing the interface that appears when a particular SNP object of the selected category is selected, according to an illustrative embodiment;

FIG. 3F is a screenshot of the GUI showing further additional information that can be viewed by scrolling when the particular SNP object is selected, according to an illustrative embodiment;

FIG. 3G is a screenshot of the GUI showing further additional information that can be viewed by scrolling further when the particular SNP object is selected, according to an illustrative embodiment;

FIG. 3H is a screenshot of the GUI showing a summary of the category that appears when the information (“i”) button of FIG. 3D is selected, according to an illustrative embodiment;

FIG. 4A is a block diagram showing a process for creating gene objects comprising one or more SNP objects using a graphical user interface, according to an illustrative embodiment;

FIG. 4B is a screenshot of a graphical user interface element for creating a new gene object using graphical control elements, according to an illustrative embodiment;

FIG. 4C is a screenshot of a user interface for adding a SNP reference and associated variants to a gene object, according to an illustrative embodiment;

FIG. 5A is a block diagram showing a process for creating categories comprising one or more SNP objects using a graphical user interface, according to an illustrative embodiment;

FIG. 5B is a screenshot of a graphical user interface element for creating a category, according to an illustrative embodiment;

FIG. 6A is a block diagram showing a process for creating products comprising one or more categories using a graphical user interface, according to an illustrative embodiment;

FIG. 6B is a screenshot of a graphical user interface element for creating a personal genetic profile product, according to an illustrative embodiment;

FIG. 7 is a screenshot of a graphical user interface showing data associated with a gene object, according to an illustrative embodiment;

FIG. 8 is a block diagram showing a process for creating a personal genetic profile assessment, according to an illustrative embodiment;

FIG. 9 is portion of a text file comprising genotyping data, according to an illustrative embodiment;

FIG. 10 is a block diagram of an example network environment for use in the methods and systems described herein, according to an illustrative embodiment; and

FIG. 11 is a block diagram of an example computing device and an example mobile computing device, for use in illustrative embodiments of the invention.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### Definitions

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context and except where such number would exceed 100% of a possible value.

Variant: As used herein, the terms “variant” refers to a specific variation of a specific SNP occurring in the genetic material of a population. In certain embodiments, a variant is a specific combination of a first allele of a first copy of an individual's genetic material (e.g. corresponding to an individual's paternal DNA) and a second allele of a second copy of an individual's genetic material (e.g. corresponding to an individual's maternal DNA), as occurs in diploid organisms (e.g. humans).

Qualifier: As used herein, the term “qualifier” refers to a classification (e.g. a label) of a particular variant of a given SNP. The qualifier associated with a given variant is the particular classification (e.g. label) of that variant. For example, a given variant may be associated with a particular qualifier of a predefined set of possible qualifiers. For example, a given variant may be associated with a qualifier selected from a group of labels such as “Adapt,” “Normal,” and “Gifted.” In certain embodiments, for a given variant of a given SNP, a qualifier corresponds to a classification of the given variant based on (i) the prevalence of the given variant within a population (e.g. if the variant is common, e.g. if the variant is rare) and/or (ii) a health-related phenotype associated with the variant. For example, a common variant may be associated with the qualifier “Normal”. A rare variant that confers a disadvantageous phenotype, such as a predisposition to high cholesterol, may be associated with the qualifier “Adapt” (e.g. classified as rare and disadvantageous). A rare variant that confers an advantageous phenotype, such as a predisposition to lower cholesterol, may be associated with the qualifier “Gifted” (e.g. accordingly, the variant is classified as rare and advantageous).

Variant object: As used herein, the term “variant object” refers to a data structure corresponding to (e.g. that is used to represent) a specific variant of a physical SNP and/or gene within a given genome (e.g., the genome of a human).

SNP object: As used herein, the term “SNP object” refers to a data structure corresponding to (e.g. that is used to represent) a specific single nucleotide polymorphism (SNP). In certain embodiments, a SNP object comprises a SNP reference that identifies the specific SNP to which the SNP object corresponds. The SNP reference may be an alphanumeric code such as an accepted name of the SNP or other identifying mark or label capable of being stored electronically. The SNP reference may be an alphanumeric code such as a National Center for Biotechnology Information (NCBI) database reference number.

Gene object: As used herein, the term “gene object” refers to a data structure corresponding to (e.g. that is used to represent) a specific physical gene within a given genome (e.g. the human genome).

Genotyping data: As used herein, the term “genotyping data” refers to data obtained from measurements of a genotype. Measurements of a genotype performed on a biological sample identify the particular nucleotide(s) (also referred to as “bases”) that is/are incorporated at one or more particular positions in genetic material extracted from the biological sample. Accordingly, genotyping measurements for a particular individual are measurements performed on a biological sample of from the individual, and which identify the particular nucleotides present at one or more specific positions within their genome.

Genotyping data may be measurements of particular genes (e.g., portions of an individual's genetic sequence, e.g., DNA sequence), or SNPs. For example, a genotyping measurement of a particular SNP for an individual identifies the particular variant of that SNP that the individual has. A genotyping measurement of a particular gene for an individual identifies the particular nucleotides that are present at one or more locations within and/or in proximity to the gene for the individual. For example, genotyping measurements of a particular gene may identify the particular variants of one or more SNPs associated with a particular gene.

In certain embodiments, genotyping data is obtained from a multi-gene panel. In certain embodiments, genotyping data is obtained from assays (e.g., TaqMan™ assays) that detect one or more specific variants of specific SNPs. In certain embodiments, genotyping data is obtained from genetic sequencing measurements. In certain embodiments, genotyping data is generated in response to a purchase or request by an individual. In certain embodiments, genotyping data comprises data for a portion of a genotype (e.g., of an individual). In certain embodiments, genotyping data comprises all available measurements of a genotype (e.g., of an individual).

Category: As used herein, the term “category” refers to a data structure corresponding to (e.g. that is used to represent) a particular health-related trait or characteristic.

Product, Genetic Profile Product, Personal Genetic Profile Product: As used herein, the terms “product,” “genetic profile product,” and “personal genetic profile product,” refer to a data structure corresponding to (e.g. that is used to represent) a general class of health-related traits and/or characteristics. In certain embodiments a product is associated with one or more categories that correspond to health-related traits and characteristics related to the general class of health-related traits and characteristics to which the product corresponds.

User: As used herein, the term “user” refers to a person, company, or organization that uses a graphical user interface to create data structures. In certain embodiments, a user also genotypes a biological sample in response to an assessment corresponding to a product being purchased or made accessible to an individual.

Individual: As used herein, the term “individual” refers to a person who uses an assessment graphical user interface in order to view information about a genome. The individual may supply one or more biological samples to be genotyped in order for a personal genetic profile assessment to be formed. The individual may purchase or be given access to one or more products in order to view a personal genetic profile assessment.

Graphical Control Element: As used herein, the term “graphical control element” refers to an element of a graphical user interface element that may be used to provide user and/or individual input. A graphical control element may be a textbox, dropdown list, radio button, data field, checkbox, button (e.g., selectable icon), list box, or slider.

Associate, Associated with: As used herein, the terms “associate,” and “associated with,” as in a first data structure is associated with a second data structure, refer to a computer representation of an association between two data structures or data elements that is stored electronically (e.g. in computer memory).

Provide: As used herein, the term “provide”, as in “providing data”, refers to a process for passing data in between different software applications, modules, systems, and/or databases. In certain embodiments, providing data comprises the execution of instructions by a process to transfer data in between software applications, or in between different modules of the same software application. In certain embodiments a software application may provide data to another application in the form of a file. In certain embodiments an application may provide data to another application on the same processor. In certain embodiments standard protocols may be used to provide data to applications on different resources. In certain embodiments a module in a software application may provide data to another module by passing arguments to that module.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

The systems and methods described herein relate to a scalable framework that provides for and facilitates the creation of personal genetic profile products. A graphical user interface allows a user to create new data structures, such as products, categories, gene objects, and SNP objects, as well as establish associations between existing data structures. The framework for creating personal genetic profile products allows previously stored data structures to be linked to newly created data structures and newly added data structures to be linked to existing data structures in order to create new products or expand existing products without requiring repetitive addition of pre-existing data structures. Products may be updated or created in response to increased interest from individuals in new categories of biological characteristics and/or traits or as new associations between SNPs and/or genes and biological characteristics and traits are developed in research. The use of scalable frameworks simplifies the effort required to update and create new products.

Data corresponding to a plurality of genotyping measurements comprising measured SNPs and measured genes, derived from one or more biological samples supplied by an individual, can be associated with related SNP objects and gene objects of one or more products to form a personal genetic profile assessment. An individual may then purchase or be given access to particular products in order to ascertain more information about his/her individual genome and potential implications for various biological characteristics and/or traits. For example, a product that relates generally to food can show an individual whether he/she is at risk for severe food allergies, more likely to find the taste of certain foods bitter, or likely to be lactose intolerant. In this way, the product is a generic data structure generalized to cover all individuals that may purchase that product, wherein the product can show an individual aspects of his/her genome when it becomes associated with the individual's genotyping data.

In some embodiments, an individual purchasing or being given access to a product prompts genotyping of the relevant genes/SNPs using the one or more biological samples supplied by the individual. In some embodiments, genotyping of genes/SNPs corresponding to the particular product purchased or made accessible can be completed by a user in approximately or under one day (e.g., 24 hours or within one business day). Such selective genotyping can allow a reduction in the expense of products to individuals as well as a reduction in the amount of stored genotyping data. Given that genetic information is highly confidential, reduced genotyping data storage can reduce liabilities and/or risks to the user and the individual. In some embodiments, the genotyping is performed using a PCR-based SNP genotyping assay. In some embodiments, genotyping is performed using genetic sequencing.

### A. Flexible Data Structure Framework

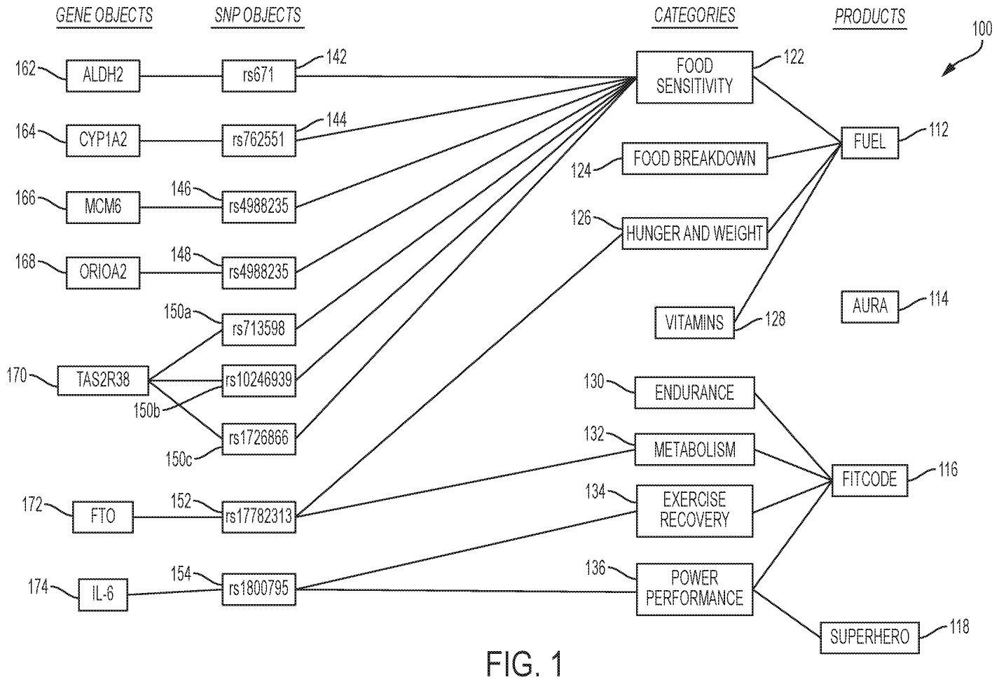

Turning to FIG. 1, in certain embodiments, in order to provide an individual not only with their personal genetic profile assessment, but also convey information related to the particular traits and characteristics that are influenced by the specific SNP variants present in their genetic material in an organized and intuitive fashion, the systems and methods described herein provide a framework comprising an intuitive hierarchical organization of data structures. The framework provides for storing relationships (e.g. associations) between particular SNPs, health-related traits and characteristics, and general classes of such health-related traits and characteristics, based on the specific phenotypes that each particular SNP influences.

In certain embodiments, a first class of data structures, referred to herein as products, are used to represent different general classes of health-related traits and characteristics. In certain embodiments, a product data structure corresponds to a particular assessment ordered (e.g., purchased by the individual), in which unique versions of genes and/or SNPs that an individual has that influence the particular general class of health-related traits and characteristics that the corresponding product represents are identified (e.g., via genotyping measurements). In certain embodiments, each product has a name (e.g. a product data structure comprises a name (e.g. text data representing the name)) that provides a convenient, and memorable way to refer to the product. For example, a particular product 112 (e.g. named “FUEL™”) is used to represent a class of traits corresponding to the way in which an individual's body processes different foods and nutrients. Another product 114 (e.g. named “AURA™”) is used to represent a class of traits corresponding to skin health. Another product 116 (e.g. named “FITCODE™”) is used to represent a class of traits corresponding to physical fitness. Another product 118 (e.g. named “SUPERHERO™”) is used to represent a class of traits corresponding to physical and intellectual performance. In certain embodiments, a name of a product is the same as the name under which a particular assessment is offered for sale. For example, assessments FUEL™, FITCODE™, AURA™ and SUPERHERO™ are offered for sale by Orgi3n, Inc. of Boston Mass.

In certain embodiments, each product is in turn associated with one or more of a second class of data structures, referred to as categories. In certain embodiments, each category corresponds to a particular health-related trait or characteristic (e.g. food sensitivity, food breakdown, hunger and weight, vitamins, skin uv sensitivity, endurance, metabolism, joint health, muscle strength, intelligence). In certain embodiments, the categories with which a particular product is associated each correspond to different health-related traits or characteristics that are related to the general class of health-related traits or characteristics to which the particular product corresponds (e.g. the general class of health-related traits or characteristics that the product represents). As with products, in certain embodiments, each category has a name (e.g. a category data structure comprises a name (e.g. text data representing the name)) that provides a convenient, and memorable way to refer to the product.

In turn, each category is associated with one or more SNP objects, each SNP object corresponding to a specific SNP. Each SNP object associated with a particular category corresponds to a specific SNP that influences a specific health related phenotype that relates to the trait or characteristic to which the particular category corresponds. Each SNP object may identify the specific SNP to which it corresponds via a SNP reference that the SNP object comprises. The SNP reference may be an alphanumeric code such as an accepted name of the SNP or other identifying mark or label capable of being stored electronically. The SNP reference may be an alphanumeric code such as a National Center for Biotechnology Information (NCBI) database reference number.

For example, the schematic of FIG. 1 shows an example of series of products, categories, and SNP objects that are associated with each other. Associated gene objects, to be described in the following, are also shown. The different products and categories are identified by their particular names, and the SNP objects each are identified by a respective SNP reference each comprises. In the example of FIG. 1, the SNP references are NCBI database reference numbers.

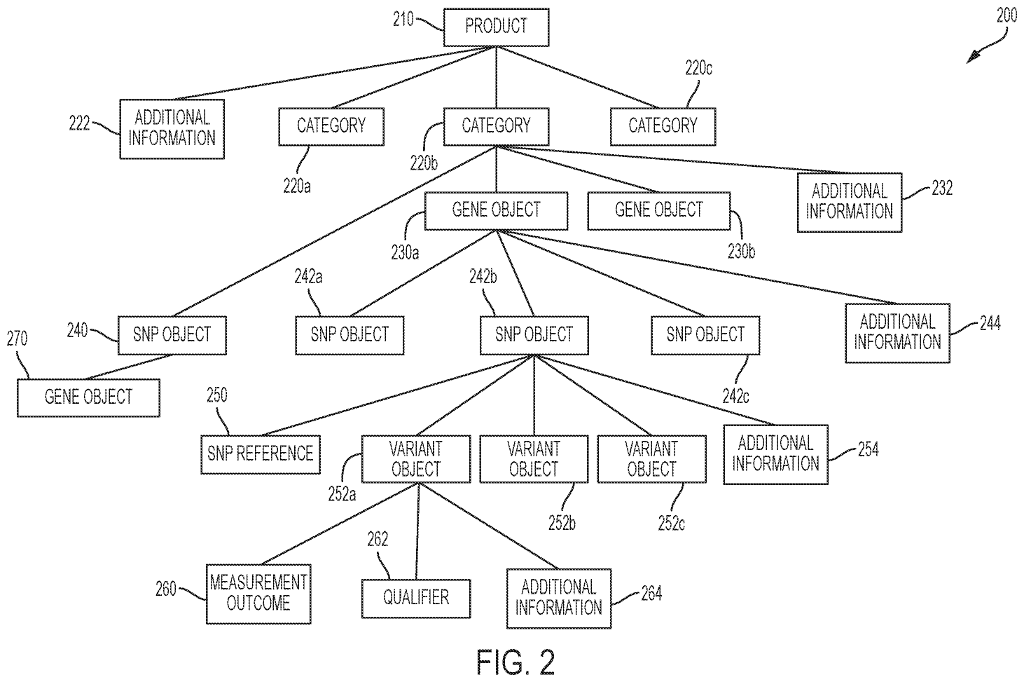

The “FUEL™” product 112 is associated with categories such as “Food Sensitivity” 122, “Food Breakdown” 124, “Hunger and Weight” 126, and “Vitamins” 128. Several SNP objects corresponding to specific SNPs that influence phenotypes related to an individual's sensitivity to different types of foods, and, accordingly, are associated with the “Food Sensitivity” category 122 are shown. In FIG. 2B, the lines connecting the SNP objects to different categories indicate the association of each particular SNP object with one or more different categories.

For example, SNP object 132 corresponds to the rs671 SNP, which influences the manner in which an individual processes alcohol. In particular, depending on the particular variant of the rs671 SNP that an individual has, the individual may process alcohol normally, or be impaired in their ability to process alcohol, and likely suffer from adverse effects resulting from alcohol consumption, such as flushing, headaches, fatigue, and sickness. Accordingly, providing an individual with knowledge of the particular variant of the rs671 SNP that they have may allow them to modify their behavior accordingly, for example, by being mindful of the amounts of alcohol that they consume (e.g. on a regular basis, e.g. in social settings).

Other SNP objects corresponding to SNPs that influence food sensitivity related phenotypes, and, accordingly, are associated with the “Food Sensitivity” category 222 are shown. For example, SNP object 144 corresponds to the rs762551 SNP that influences caffeine metabolism, SNP object 146 corresponds to the rs4988235 SNP that influences lactose intolerance, and SNP object 148 corresponds to the rs72921001 SNP that influences an aversion to the herb Cilantro (e.g. depending on the particular variant of this SNP that an individual has, they may either perceive Cilantro as pleasant tasting, or bitter and soap-like in taste).

In certain examples, multiple SNPs are associated with a particular phenotype and, accordingly, the SNP objects to which they correspond may be grouped together. For example, three SNPS—rs713598 (corresponding to SNP object 150a), rs10246939 (corresponding to SNP object 150b), and rs1726866 (corresponding to SNP object 150c),—influence the sensitivity of an individual to bitter tasting foods (e.g. cabbage, broccoli, cauliflower, kale, brussel sprouts, and collard greens), and, accordingly, their enjoyment of or aversion to such foods.

SNPs correspond to specific locations within or nearby (e.g. a SNP may occur in a promotor region that influences transcription of a particular gene, e.g. a SNP may occur within 5 kb upstream or downstream of a particular gene, e.g. a SNP may occur within 100 kb upstream or downstream of a particular gene, e.g. a SNP may occur within 500 kb upstream or downstream of a particular gene, e.g. a SNP may occur within 1 Mb upstream or downstream of a particular gene) genes in an individual's genetic material. Accordingly, in certain embodiments, as shown in FIG. 1, each SNP object is associated with a gene object that corresponds to the particular gene within or nearby to which the SNP to which the SNP object corresponds is present. For example, the rs671 SNP corresponds to a location within the ALDH2 gene; the rs762551 SNP corresponds to a location within the CYP1A2 gene, the rs4988235 SNP occurs within the MCM6 gene, and the rs72921001 SNP occurs within the OR10A2 gene. Accordingly, SNP object 142 (corresponding to the rs671 SNP) is associated with gene object 162 (corresponding to the ALDH2 gene). Similarly, SNP object 144 (corresponding to the rs762551 SNP) is associated with gene object 162 (corresponding to the CYP1A2 gene), SNP object 146 (corresponding to the rs4988235 SNP) is associated with gene object 166 (corresponding to the MCM6 gene) and SNP object 148 (corresponding to the rs72921001 SNP) is associated with gene object 168 (corresponding to the OR10A2 gene).

Other SNPs objects correspond to SNPs that are nearby particular genes of interest and thereby influence phenotypes associated expression of the gene. For example, rs12696304 is a SNP that lies 1.5 kb downstream from the TERC gene, and influences biological aging associated with the TERC gene. Accordingly, in one example, a SNP object corresponding to the rs12696304 SNP is associated a gene object corresponding to the TERC gene.

In certain embodiments, multiple SNPs of interest occur within a single gene. For example, the three SNPs related to bitter taste—rs713598, rs10246939, and rs1726866—occur within the TAS2R38 gene. Accordingly, SNP objects 150a, 150b, and 150c, which correspond to the rs713598, rs10246939, and rs1726866 SNPs, respectively, are all associated with a gene object 170 corresponding to the TAS2R38 gene.

In certain embodiments, different products correspond to different general classes of health-related traits and characteristics. For example, products may be based on particular organs (e.g. product 114, named “AURA™”, is related to skin health), or particular habits, activities, or bodily functions. For example, food related biological characteristics and traits may be covered by a single products or a plurality of products. A single product or a plurality of products may be based on learning and brain function characteristics and traits. A single product or a plurality of products may be based on physical fitness (e.g., cardiovascular strength, agility, flexibility, muscular strength).

For example, as shown in FIG. 1, another product 116 (e.g. named “FITCODE™”), relates to a general class of physical fitness related traits, and, accordingly, comprises categories associated with endurance 130 (“Endurance”), metabolism 132 (“Metabolism”), the ability of an individual to recover effectively following exercises 134 (“Exercise Recovery”), and cardiovascular fitness and skeletal muscle makeup 136 (“Power Performance”).

In certain embodiments, a particular SNP object is associated with two or more categories. For example, the rs17782313 SNP, occurring in the FTO gene, influences an individual's appetite. Accordingly, as shown in FIG. 1, the SNP object 152 corresponding to the rs17782313 SNP is associated with both the “Hunger and Weight” category 126 of the “FUEL™” product, and the “Metabolism” category 132 of the “FITCODE™” product. SNP object 152 is also associated with gene object 172, reflecting the fact that the rs17782313 SNP occurs in the FTO gene. In certain embodiments, as with the rs17782313 SNP object, each of a first category and a second category with which a particular SNP object is associated are associated with a different product. In certain embodiments, a particular SNP object is associated with a first category and a second category, and both the first category and the second category are associated with the same product.

For example, the SNP object 154 corresponding to the rs1800795 SNP of the IL-6 gene (accordingly, SNP object 154 is associated with gene object 174, which corresponds to the IL-6 gene) is associated with the “Exercise Recovery” category 134 and the “Power Performance” category 136, both of which are associated with the “FITCODE™” product 116. In addition, in certain embodiments, a category is associated with two or more products. For example, the “Power Performance” category 136 is associated with the “FITCODE™” product 116, as well as the “SUPERHERO™” product 118, which provides an assessment of a general class of traits related to physical and intellectual performance.

Thus, by providing a framework comprising a hierarchical organization of data structures corresponding to products, categories, SNP objects, and gene objects, the systems, methods, and architectures described herein provide an intuitive and flexible approach to storing, updating, and creating new associations between different classes of health-related traits and characteristics, and the underlying genetic variations corresponding to different specific SNPs that influence them.

In certain embodiments the hierarchical organization of product, category, SNP object and gene object data structures serves as a flexible template that facilitates both the rapid creation of individual personal genetic profile assessments from genotyping measurements taken from a plurality of individuals, and the presentation of an individual's personal genetic profile assessment. In particular, an individual may purchase assessments corresponding to different products, in order to gain insight into the manner in which their personal genome influences the different general classes of health-related traits and characteristics to which each different product corresponds. Accordingly, an individual's personal genetic profile assessment corresponding to one or more products comprises, for each specific SNP associated with each category that is associated with each of the one or more products, an identification of the particular variant of the specific SNP that the individual has. Typically, the identification is obtained via one or more genotyping measurements performed on a biological sample taken from the individual (e.g. a blood sample, e.g. a cheek swab sample, e.g. a saliva sample).

In certain embodiments, an individual may purchase a first assessment corresponding to a first product, and provide a biological sample for genotyping. The individual's biological sample may be stored (e.g. cryogenically frozen). After a period of time, the individual may choose to purchase additional assessments corresponding to other products, and the individual's previously stored biological sample may be taken from storage for additional genotyping measurements of the additional SNPs that are associated with the new products. Moreover, in certain embodiments, additional new products may be created over time, and new assessments corresponding to new products offered to and purchased by individuals. In certain embodiments, as new information related to the influence of new and/or existing SNPs on different specific health related phenotypes is elucidated, new SNP objects and gene objects may be created, and new associations between them and new or existing categories and/or products established. In certain embodiments, existing personal genetic profile assessments of individuals are automatically updated to reflect new information.

In certain embodiments, in order to facilitate the creation and presentation of individual personal genetic profile assessments (e.g. corresponding to one or more different products) based on the framework described above, the product, category, SNP object, and gene object data structures described herein store a variety of information. FIG. 2 is a block diagram of a hierarchy of data structures 200 of an example genetic profile product. An exemplary data structure of each type is shown to be associated with sub-data structures in order to simplify presentation of the figure. It is understood that data structures may be associated to any number of other data structures in the hierarchy if the association is consistent with the associations shown in FIG. 2. For example, category 220b is associated with gene objects 230a-b while category 220c may be associated with one or more gene objects and/or SNP objects, but any such associations are not shown. In some embodiments, data structures may be created without also forming associations between other structures of relevant types. For example, unassociated or partially associated data structures may be created for planning purposes such as during product or category development (e.g., category 220a has no associations yet because its scope has not been determined yet by the user). For example, unassociated or partially associated data structures may be created to allow genotyping data to be associated with relevant gene objects or SNP objects in order to retain the data in a ready to use format in the event that the gene objects and/or SNP objects are later associated with one or more categories.

Referring now to FIG. 2, product 210 comprises three categories 220a-c and additional information 222. Additional information 222 may be a name of the product, an icon associated with the product, and/or a description of the product. Category 220b comprises two gene objects 230a-b, one SNP object 240, and additional information 232. Additional information 232 may comprise a name of the category, a background image associated with the category, an icon associated with the category, a category order identifier, and/or a description of the category. SNP object 240 is associated with gene object 270. Gene object 230a is associated to three SNP objects 242a-c. Categories may be associated directly to SNP objects, such as category 220b is associated with SNP object 240, or they may be associated indirectly such as SNP objects 242a-c are associated to category 220b via gene object 230a. The ability to form associations indirectly allows all SNP objects associated with a particular gene object to be associated with a category by forming a single association in cases where all SNP objects of a particular gene are relevant to a particular category. The ability to form associations directly allows a particular SNP object to be associated with a category without also forming an association with all other SNP objects associated with the gene object associated with the particular SNP object in cases where only one or a subset of SNP objects of a particular gene object are relevant to a category.

Gene object 230a is also associated with additional information 244. Additional information 244 may comprise one or more data structures comprising information such as a unique gene identifier that corresponds gene object 230a to a specific physical gene and descriptive information about the corresponding gene. The gene identifier may be an alphanumeric code such as an accepted name of the gene or other identifying mark or label capable of being stored electronically. Additional information may be stored as a single data structure or a plurality of data structures.

SNP object 242b is associated with SNP reference 250, and additional information 254. SNP reference 250 is a unique identifier of the SNP that corresponds the SNP object to a specific physical SNP. The SNP reference may be an alphanumeric code such as an accepted name of the gene or other identifying mark or label capable of being stored electronically. The SNP reference may be an alphanumeric code such as a National Center for Biotechnology Information (NCBI) database reference number. Additional information 254 may comprise one or more data structures with other descriptive information about the corresponding SNP.

Variants of a particular SNP can be represented within a corresponding SNP object using various combinations of data elements such as a measurement outcomes, and qualifiers. For example, a particular variant of a SNP can be identified by a measurement outcome, which is an identifier, such as an alphanumeric code, that identifies the specific alleles corresponding to the particular variant. For example, a measurement outcome such as the string “CC” identifies a first variant of the rs762551 SNP in which an individual has a cytosine (C) at the rs762551 position in each copy of their genetic material. A measurement outcome such as the string “AC” identifies a second variant of the rs762551 SNP in which an individual has a C in one copy and an adenine (A) in the other at the rs762551 position. A measurement outcome such as the string “AA” identifies a second variant of the rs762551 SNP in which an individual has an A at the rs762551 position in each copy of their genetic material. A qualifier is an identifier, such as an alphanumeric code, that identifies a classification of a variant, wherein the classification may be based on the prevalence of the variant within a population, a health-related phenotype associated with the variant, and/or other relevant classification bases. Additional information may also be included within a SNP object to describe a particular variant.

In certain embodiments, measurement outcomes and qualifiers that identify and classify, respectively the same variant are associated with each other to form a variant object associated with the SNP object. For example, variant object 252a comprises measurement outcome 260, qualifier 262. Variant object 252a is also comprises additional information 264. Additional information 264 comprises a description of the variant. For example, the additional information comprises a description of the specific health-related phenotype that an individual with the variant represented by variant object 252a exhibits or an explanation of the prevalence of the variant. A SNP object may be associated with a variant object to represent each variant of the particular SNP to which it corresponds. For example, SNP object is associated with three variant objects 252a-c.

### B. Presentation of Individual Personal Genetic Profile Assessments

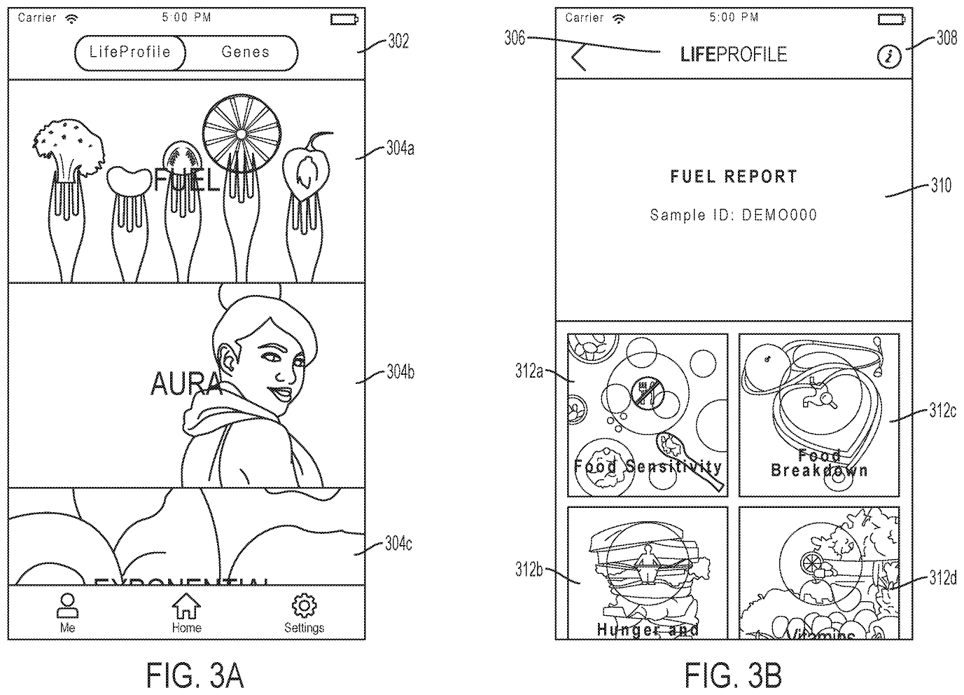

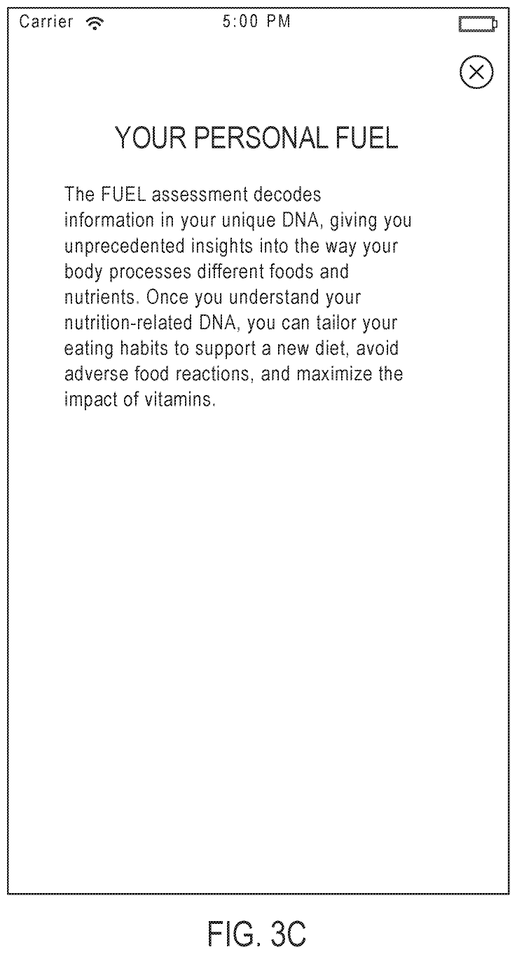

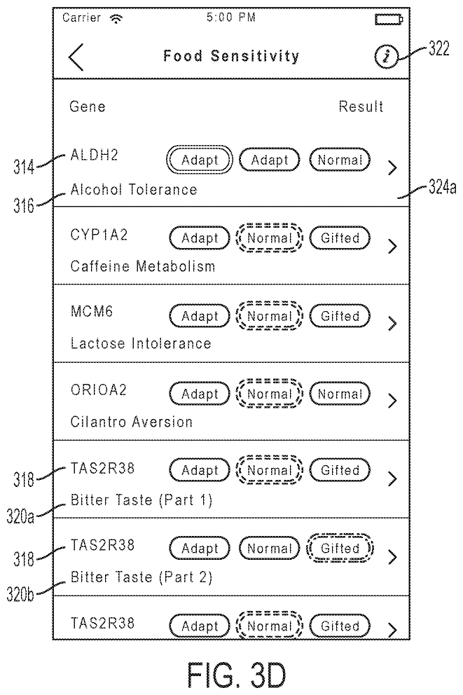

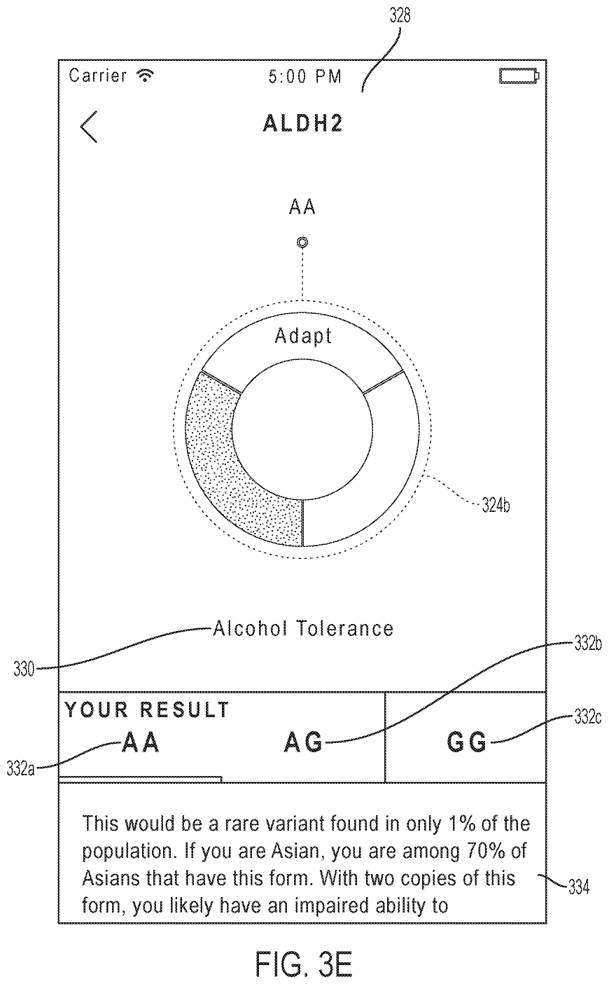

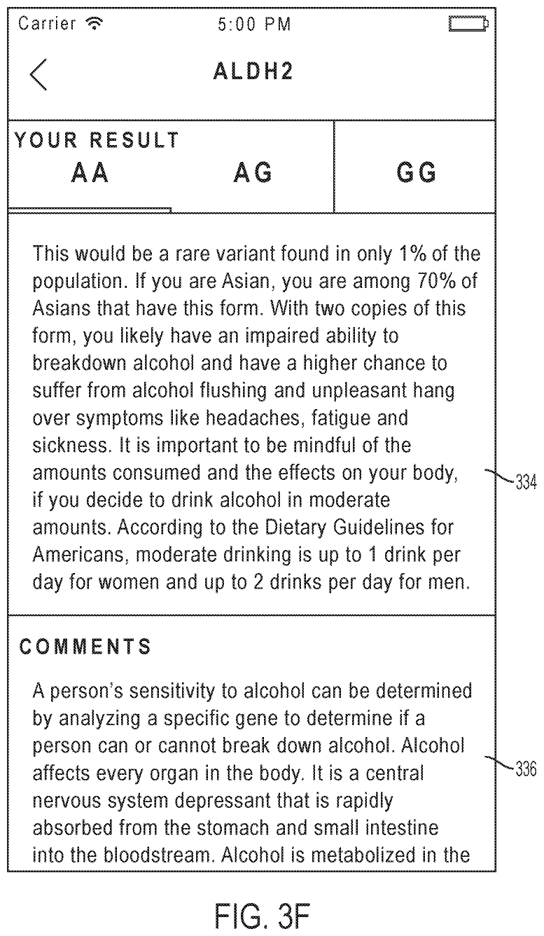

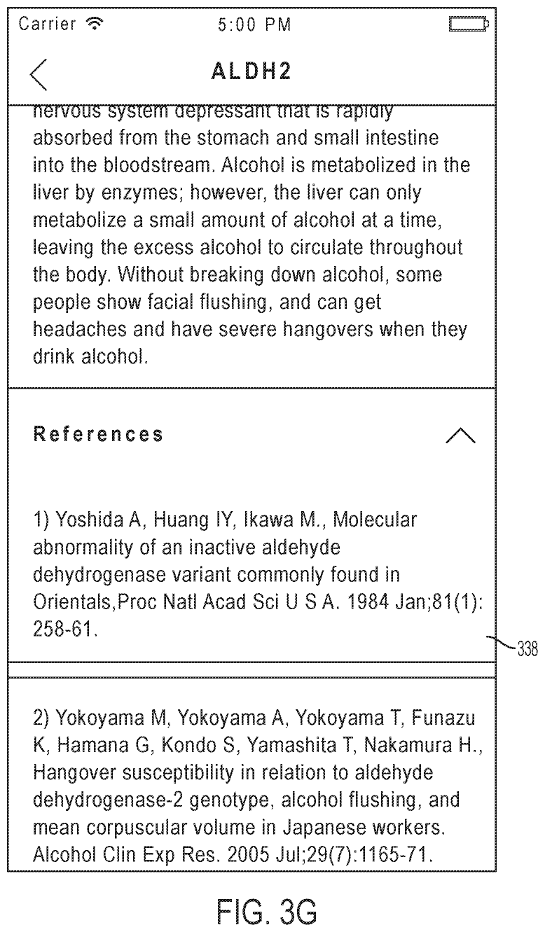

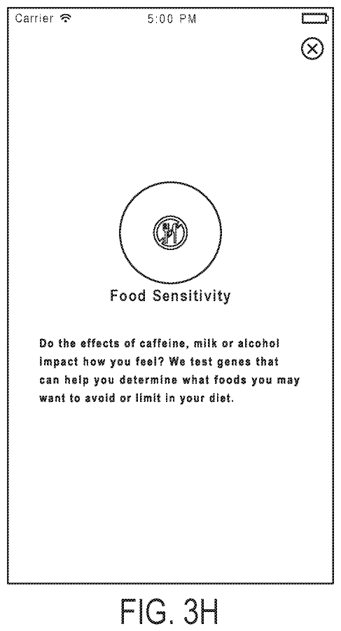

In certain embodiments, an individual views their genomic information using an assessment graphical user interface (assessment GUI) that is populated using one or more products (e.g., one or more hierarchies of data structures, such as the exemplary hierarchy of FIG. 2) and the individual's personal genetic profile assessment. In certain embodiments, the individual's personal genetic profile assessment is associated with the one or more products using a plurality of associations such that the assessment GUI is populated using the plurality of associations. In some embodiments, the one or more products are personalized by updating the hierarchies of data structures to comprise the data of the individual's personal genetic profile assessment such that the assessment GUI is populated using one or more products modified to be personalized to the individual. The assessment GUI allows individuals to interactively view their genomic information by navigating through the layers of data structures from the product level down to the level of information for individual SNPs. FIGS. 3A-3H are snapshots of an exemplary assessment graphical user interface that an individual would use to view their genomic information.

Referring now to FIG. 3A, the screenshot shows a home screen an individual uses to navigate to specific information about their personal genetic profile assessment. Three products 304a-c are visible: “FUEL™” 304a, “AURA™” 304b, “EXPONENTIAL™” 304c. Each product corresponds to a different set of genes that determine biological characteristics and traits. Selector 302 allows the individual to switch between his/her “LifeProfile™” that allows for navigation to specific information through the hierarchy of data structures, whereas Genes allows the individual to scroll through a listing of all SNPs corresponding to products that the individual has purchased or been given access to.

In certain embodiments, an assessment graphical user interface also includes a graphical control element for sharing data corresponding to an individual's personal genetic profile assessment (or a portion thereof) with one or more sharing entities. Sharing entities may be other individuals, people, or services with whom an individual wishes to share. For example, an individual may wish to share his or her entire personal genetic profile assessment with a friend, a spouse, or a social media service. Upon selection of the graphical control element for sharing, one or more graphical control elements may be provided for selecting which portions (e.g., products, categories, or a selected list of individual SNPs and/or genes) of a personal genetic profile assessment an individual would like to share (e.g., in the event that the individual would prefer certain portions of his or her personal genetic profile assessment remain private). In certain embodiments, the data is in a PDF report generated from the individual's personal genetic profile assessment.

Selecting a graphical control element for sharing may provide an individual with additional graphical control elements with which to select exactly with whom and by what method a personal genetic profile assessment (or portion thereof) is shared. For example, graphical control elements may be provided for selecting whether to text, email, or post the personal genetic profile assessment (or portion thereof) and other graphical control elements may be provided to allow an individual to select one or more recipients from among his contacts or enter contact information such as a phone number or email address. For example, an individual may select only certain friends or followers on a social media site with whom the personal genetic profile assessment (or portion thereof) is shared.

In certain embodiments, an individual uses a graphical control element for sharing in order to give access to other individuals using an assessment graphical user interface for viewing information about their genomes. For example, a first individual may use a graphical control element for sharing to give access to their personal genetic profile assessment to a spouse, wherein the spouse views the first individual's personal genetic profile assessment using an assessment GUI. In certain embodiments, an assessment GUI includes a graphical control element that an individual uses to select whose personal genetic profile assessment he or she is viewing. For example, the spouse may use such a graphical control element to toggle between viewing his or her own personal genetic profile assessment and the first individual's personal genetic profile assessment.

By selecting the “FUEL™” product from the LifeProfile™ listing of FIG. 3A, the individual sees the assessment GUI state of FIG. 3B. LifeProfile™ indicator 306 reminds the individual that he/she is using the LifeProfile™ navigation system. The information button 308 can be selected to view the brief description associated with the “FUEL™” product in its data hierarchy, as shown in FIG. 3C. Referring again to FIG. 3B, Fuel Report 310 provides space for a summary of the individual's “FUEL™” product genomic information. Categories 312a-d may be selected to view specific genomic information regarding different aspects of the individual's genome related to food and eating (e.g., different aspects of the “FUEL™” product). For each of the four categories, the individual sees the name of the category, the background image associated with the category, and the icon associated with the category. For example, category 312a is named Food Sensitivity, where the icon is a fork and knife with a slash, and the background image shows a variety of foods on a table.

Selecting the Food Sensitivity category 312a brings the individual to a view of the assessment GUI shown in FIG. 3D. The information button 322 can be selected to view the brief description associated with the Food Sensitivity category, as shown in FIG. 3H. Referring again to FIG. 3D, the individual may scroll through a list of selectable control elements corresponding to each of the SNPs related to the category, wherein each selectable control element comprises brief summary information that the individual may use to determine which selectable control element to select. For example, the first selectable control element in the list shown in FIG. 3D comprises a short description of a SNP 316, a graphical representation of a gene identifier 314 corresponding to the SNP, and a graphical representation of the qualifier 324a associated with the variant corresponding to the individual's particular alleles of the SNP.

The short description of the SNP 316 characterizes the biological characteristic or trait influenced by the corresponding SNP in the individual's genome. For example, the short description of the SNP 316 is “Alcohol Tolerance.” The individual would see that selecting the first selectable control element in the list would provide the individual with information about how the individual's genome influences his/her tolerance for alcohol consumption. The individual may select a particular selectable control element to view detailed information based on the short description of a SNP and/or the qualifier of the variant corresponding to his/her particular alleles of the SNP (as displayed by the graphical representation of the qualifier).

The graphical representation of the qualifier 324a is a graphic showing each of the qualifiers associated with the three variants corresponding to the SNP with the qualifier of the particular variant corresponding to the individual's alleles highlighted. Qualifiers may be words or short phrases that characterize the variant. For example, “adapt” may be used to characterize variants that are uncommon and/or disadvantageous; “normal” may be used to characterize variants that are common and/or neither advantageous nor disadvantage; and “gifted” may be used to characterize variants that are uncommon and/or advantageous. The graphical representation of the qualifier 324a highlights the qualifier associated with the variant corresponding to the individual's alleles in red. Different colors may be used to highlight different qualifiers in a graphical representation of a qualifier. For example, in FIG. 3D, when highlighted in a graphical representation of a qualifier, “adapt” qualifiers are highlighted in red, “normal” qualifiers are highlighted in blue, and “gifted” qualifiers are highlighted in green.

Some genes have multiple related SNPs. The related SNPs may influence a single biological characteristic or trait or a plurality of biological characteristics and/or traits. Each SNP may correspond to a unique selectable control element in the assessment GUI. For example, the graphical representation of the gene identifier 318 appears in two separate selectable control elements shown FIG. 3D since at least two unique SNPs relate to the gene corresponding to the graphical representation of the gene identifier 318. The two unique SNPs are differentiated by unique corresponding short descriptions 320a (“Bitter Taste (Part 1)”) and 320b (“Bitter Taste (Part 2)”). Short descriptions 320a and 320b correspond to related SNPs that influence an individual's sensitivity to bitterness in food.

Selecting the first selectable control element identified by short description 316 (“Alcohol Tolerance”) brings the individual to a view of the assessment GUI shown in FIG. 3H comprising detailed information regarding the SNP corresponding to the short description “Alcohol Tolerance.” The graphical representation of the gene identifier 328 is shown at the top of the screen. A graphical representation of the qualifier 324b associated with the variant corresponding to the individual's particular alleles of the SNP identified in the first selectable control element. Graphical representation 324b displays both that the measurement outcome corresponding to the individual's alleles is “AA” and that the qualifier associated with this variant is “Adapt”. The other two segments of the ring in graphical representation 324b relate to the other two variants corresponding to the SNP and are color coded to the associated qualifiers as described above. The graphical representation 324b is an alternative to the graphical representation 324a of FIG. 3D. Graphical control elements 332a-c indicate the measurement outcomes associated with each of the three variants corresponding to the SNP. Graphical control element 332a indicates that the individual's alleles correspond to the variant identified by the displayed measurement outcome (by displaying “Your Result” above the measurement outcome) as well as that information currently displayed below the row of graphical control elements 332a-c is associated with that variant (by displaying the light blue bar under the measurement outcome). A portion of description 334 associated with the variant identified in graphical representation 332a is visible. An individual may select other graphical control elements identified by other measurement outcomes to view information associated with other variants.

Referring now to FIG. 3F, the individual may scroll in order to read more information regarding their genome. By scrolling, the complete description 334 may be read as may other additional information 336, which may be include a brief description associated with a SNP object corresponding to the SNP. Scrolling further, an individual can see references 338 that provide further detail related to the currently selected variant of the SNP object, as shown in FIG. 3G.

The assessment GUI shown in FIGS. 3A-3H is configured for display on mobile devices (e.g., smartphones, tablets, PDAs), but an assessment GUI may also be configured for viewing on a computing device using the web (e.g., with a laptop or desktop computer).

The assessment GUI is populated using data associated with one or more products. A standardized graphical user interface element (e.g., widget) is used to create data and data structures as well as associations between existing and new data and data structures.

### C. Creation of Gene Objects, SNP Objects, Categories, and Products

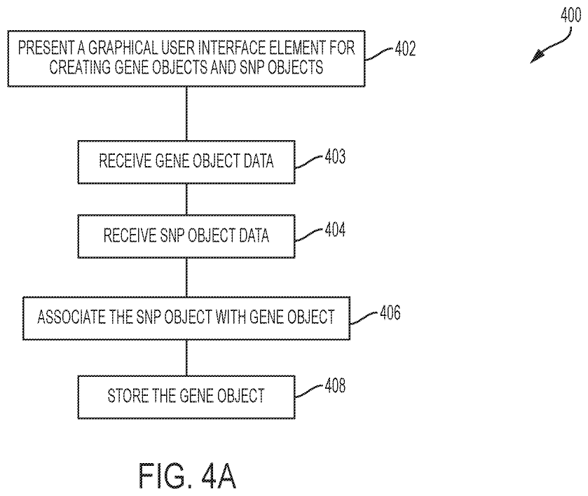

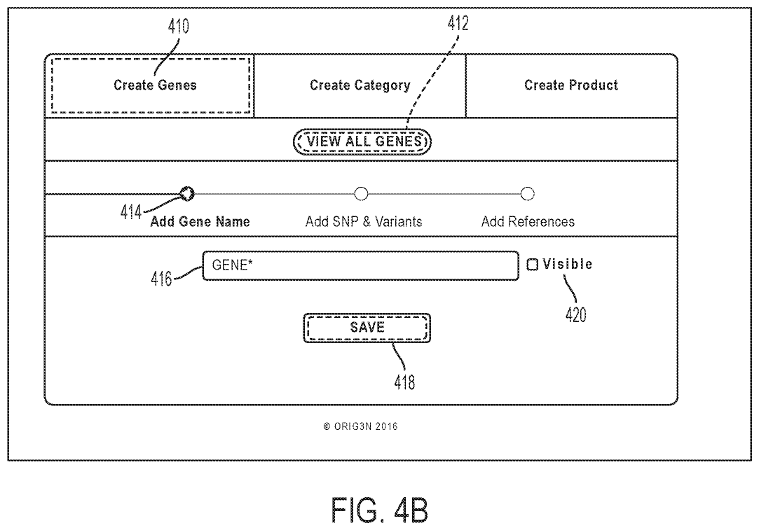

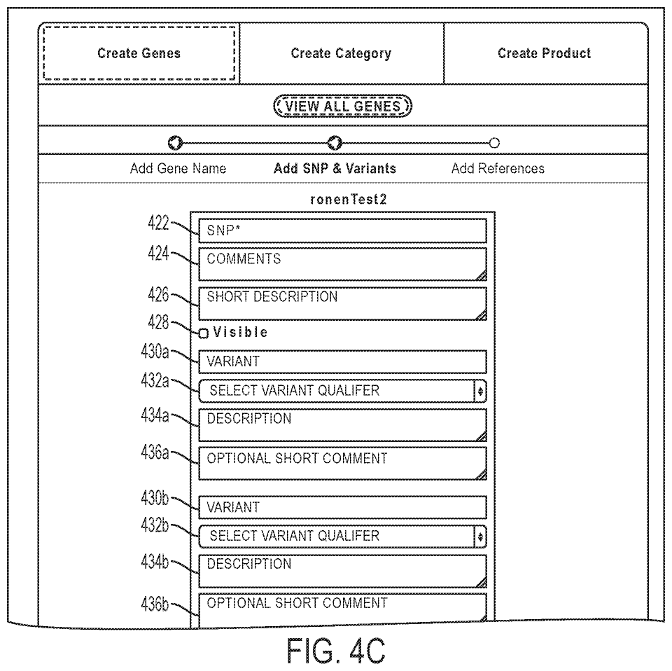

FIG. 4A is a block diagram of a process 400 for creating gene objects, SNP objects, and associations between them. In step 402, a graphical user interface element (GUI element) is presented to a user in order to create gene objects and SNP objects. The user can then input gene object data into the GUI element, which is received by the processor of a computing device in step 403. The user can then input SNP object data, which is received by the processor of the computing device in step 404. The SNP object data may be for association to a single SNP object or a plurality of SNP objects. The processor then associates the SNP object and its associated data with the gene object and its associated data in step 406. Lastly, the gene object and all associated data (which comprises all input SNP object data) are stored by the processor for future retrieval and/or updating in step 408.

Snapshots of an exemplary graphical user interface element used in process 400 are shown in FIGS. 4B-4C. Graphical control element 410 is selected to allow the user to create new gene objects and SNP objects. Graphical control element 412 can be selected to view all gene objects currently stored, for example, for reference or to select a stored gene object in order to update it. Status bar 414 indicates progress in the process or creating new genes objects and SNP objects. Graphical control element 416 provides the user an entry field for entering a gene identifier to be associated with the gene object being created. Graphical control element 420 may be selected or unselected to toggle the visibility of the gene object being created, for example, to allow a user to develop new or existing products without interfering with the use experience of individuals viewing related interfaces. The user can save the input information using graphical control element 418.

After selecting graphical control element 418, the user views the graphical control element state of FIG. 4C. Several graphical control elements allow the user to input data associated with a SNP object that is to be created. The user inputs a SNP reference using graphical control element 422. The user inputs comments to be associated with the new SNP object using graphical control element 424. The user inputs a short description to be associated with the new SNP object using graphical control element 426. The user may toggle visibility of the new SNP object using graphical control element 428.

The graphical control elements below graphical control element 428 are used to input data associated with variants that will be represented in the SNP object. For each variant to be represented, the user inputs a measurement outcome to be used to identify the variant using graphical control elements 430a-b. The user inputs qualifiers for each variant using graphical control elements 432a-b. The user inputs descriptions to be associated with the new variants using graphical control elements 434a-b. The user inputs short comments to be associated with the new variants using graphical control elements 436a-b. The measurement outcome, qualifier, description, and short comments that relate to the same variant are associated with each other and stored as a variant object that represents the variant. A graphical control element that when selected provides a further set of graphical control elements for inputting data associated with an additional variant (not shown) may additionally be provided. In certain embodiments, exactly three variants are represented in a SNP object.

A graphical control element may be provided that when selected provides graphical control elements for created additional new SNP objects with associated input data (not shown in FIG. 4C). After input of all data for the final desired SNP object, selection of a graphical control element for saving (not shown) allows the user to input reference data that will be associated with one or more of the new SNP objects.

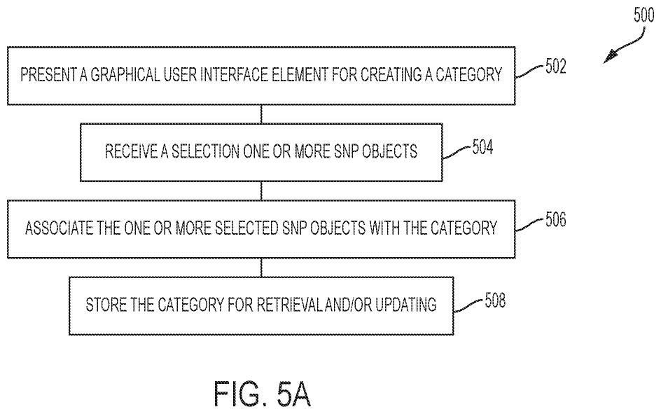

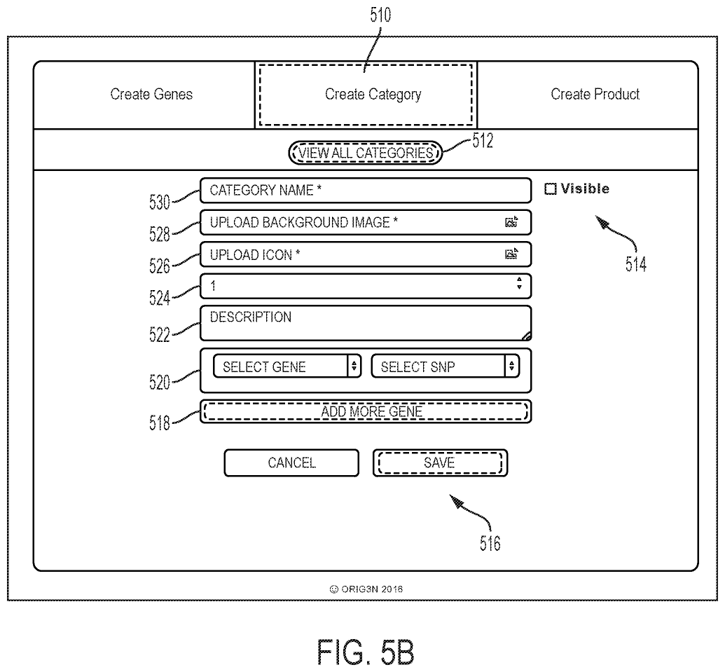

FIG. 5A is a block diagram of a process 500 for creating categories and associating existing gene objects and SNP objects with the new categories. In step 502, a GUI element is presented to a user for creating a category. In step 504, the user inputs a selection of one or more SNP objects to be received by the processor of a computing device. The processor of the computing device associates the one or more selected SNP objects with the category in step 506. Lastly, the category is stored for future retrieval and/or updating in step 508.

A snapshot of an exemplary graphical user interface element used in method 500 is shown in FIG. 5B. Graphical control element 510 is selected to allow the user to create new categories. Graphical control element 512 can be selected to view all categories currently stored, for example, for reference or to select a stored category in order to update it. Graphical control element 530 provides the user an entry field for entering a name to be associated with the category being created. Graphical control element 528 provides the user an entry field for uploading a background image to be associated with the category being created for use in displaying graphical control elements for selecting the category in an associated product. Graphical control element 526 provides the user an entry field to upload an icon to be associated with the category being created for graphically identifying the category. Graphical control element 524 provides the user a dropdown list to select an order preference for displaying the category being created for any associated products. Graphical control element 522 provides the user an entry field for entering a description to be associated with the category being created.

Graphical control element 520 provides the user two dropdown lists for selecting a SNP object to be associated with the category being created by selecting from a list of all gene objects and a list of all SNP objects. In some embodiments, the dropdown list of SNP objects only displays SNP objects associated with the gene object selected in the gene object dropdown list. The gene object and SNP object dropdown lists may display the gene identifiers and SNP references, respectively, corresponding to stored genes and SNPs in order to allow the user to select the desired data structure. In some embodiments, if the user only selects from the gene object dropdown list, all SNP objects associated with that gene object will be associated with the category being created. Only one SNP object is selected in graphical control element 520. Graphical control element 518 may be selected to provide another set of dropdown lists for adding an additional SNP object (e.g., a new copy of graphical control element 520).

Graphical control element 514 may be selected or unselected to toggle the visibility of the category being created, for example, to allow a user to develop new or existing products without interfering with the use experience of individuals viewing related interfaces. The user can save the input information using graphical control element 516.

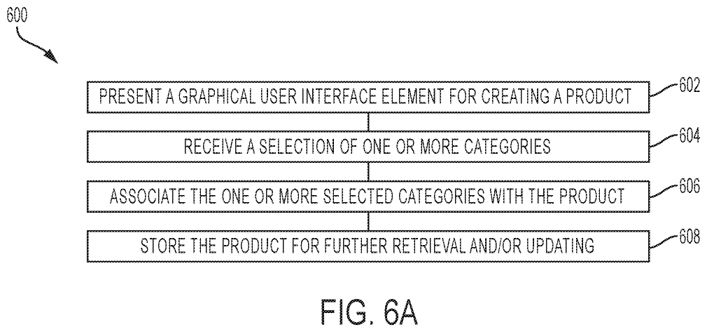

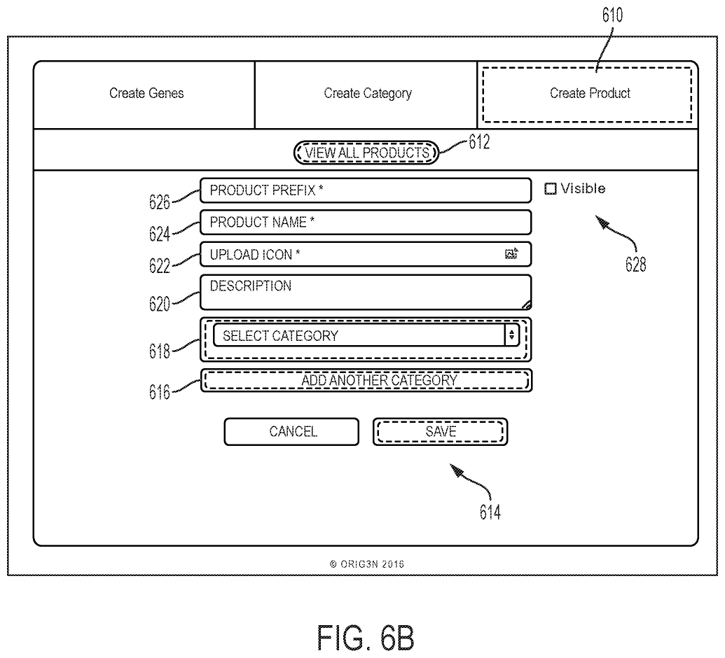

FIG. 6A is a block diagram of a process 600 for creating products and associating existing categories with the new product. In step 602, a GUI element is presented to a user for creating a product. In step 604, the user inputs a selection of one or more categories to be received by the processor of a computing device. The processor of the computing device associates the one or more selected categories with the product in step 506. Lastly, the product is stored for future retrieval and/or updating in step 508.

A snapshot of an exemplary graphical user interface element used in process 600 is shown in FIG. 6B. Graphical control element 610 is selected to allow the user to create new categories. Graphical control element 612 can be selected to view all products currently stored, for example, for reference or to select a stored category in order to update it. Graphical control element 626 provides the user an entry field for entering a prefix to be associated with the product being created. Graphical control element 624 provides the user an entry field for entering a name to be associated with the product being created. Graphical control element 622 provides the user an entry field to upload an icon to be associated with the product being created for graphically identifying the product. Graphical control element 620 provides the user an entry field for entering a description to be associated with the product being created. Graphical control element 618 allows a user to select a category to be associated with the product being created. Selection of graphical control element 616 provides an additional graphical control element for selecting an additional category to be associated with the product being created (e.g., a new copy of graphical control element 618). Graphical control element 628 may be selected or unselected to toggle the visibility of the product being created, for example, to allow a user to develop new or existing products without interfering with the use experience of individuals viewing related interfaces. The user can save the input information using graphical control element 614.

Processes 400, 500, and 600 may be performed using a single graphical user interface element or a set of graphical user interface elements. In some embodiments, new data structures (e.g., gene objects, SNP objects, categories, products) may be created using a minimally necessary set of user inputs (e.g., input from only a set of required graphical control elements). For example, a user may wish to create a new category without associating any gene objects or SNP objects. This may require entering a category name, background image, and icon with all other input being optional.

A user may wish to examine or otherwise review data associated with various data structures without having to navigate between different views of a graphical user interface in order to see relevant data. A data graphical user interface (data GUI) may be used to display all data associated with a particular data structure (e.g., object). Such a data GUI allows a user to quickly and efficiently view large amounts of data. In certain embodiments, the data GUI is configured to allow a user to select from all stored gene objects. Upon selection of a particular gene object, the data GUI displays data associated with each SNP object of the gene object, including information related to each of the variant objects (e.g. measurement outcomes, e.g. qualifiers) of each SNP object associated with the gene object.

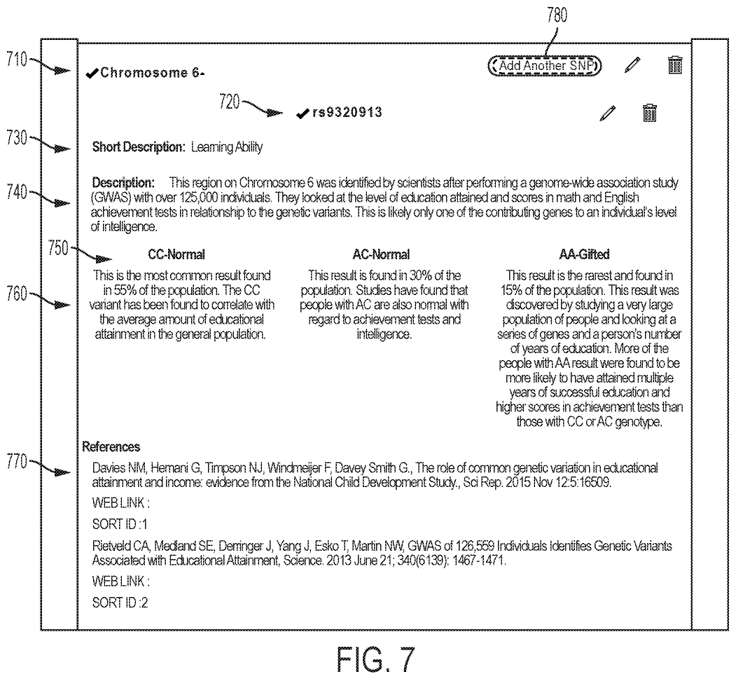

A snapshot of an exemplary data GUI is shown in FIG. 7. Data 710 identifies the gene object. A SNP object associated with the gene object is identified by SNP reference 720. Short description 730 displays the word or words used in assessment GUIs to identify a SNP object corresponding to a relevant SNP. Description 740 shows a detailed description associated with the SNP object. Text 750 identifies a particular variant object associated with the SNP object in order to represent a particular variant of the SNP represented by the SNP object. Text 750 identifies the variant by a combined display of the particular variant object's associated measurement outcome (in this case “CC”) and associated qualifier (in this case “Normal”). Description 760 shows the description associated with the particular variant object corresponding to text 750. References 770 associated with the SNP object are also shown. Graphical control element 780 may be selected to create another SNP object (e.g. associated with the gene object). Graphical control element 790 may be used to edit the SNP object identified by the data being displayed and graphical control element 795 may be used to delete the data.

### D. Automated Creation of Individual Personal Genetic Profile Assessments

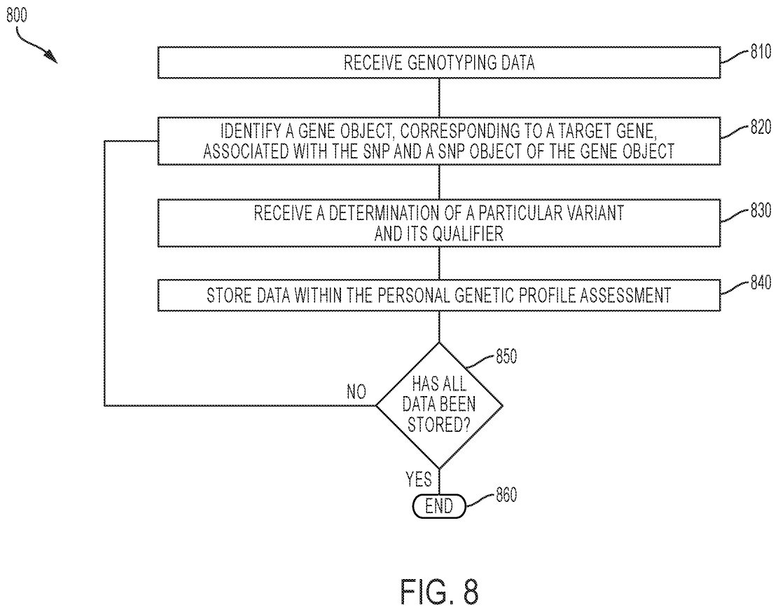

In order to populate an assessment GUI to provide to an individual, genotyping data must be added to the individual's personal genetic profile assessment. FIG. 8 is a block diagram of a method 800 for adding genotyping data to an individual's personal genetic profile assessment. In step 810, a processor of a computing device receives genotyping data. In step 820, the processor identifies a gene object corresponding to a gene measured in the genotyping data and a SNP object corresponding to a SNP associated with the gene [e.g. the SNP occurring within the gene or occurring nearby the gene (e.g. within a promotor region that influences transcription of the gene, e.g. within 5 kb upstream or downstream of the gene, e.g. within 100 kb upstream or downstream of the gene, e.g. within 500 kb upstream or downstream of the gene, e.g. within 1 Mb upstream or downstream of the gene)]. In certain embodiments, genotyping data is stored as a table of data in a text file where each row corresponds to a unique SNP. In step 830, a particular variant of SNP represented by the identified SNP object and its associated qualifier are determined based on data from genotyping measurements. For example, data corresponding to the measurement outcome of a particular variant may be stored as one or more columns at the end of each row. In step 840, the data is stored in the individual's personal genetic profile assessment. In step 850, the processor determines if all data of the genotyping data has been stored. If all data has not been stored in the individual's personal genetic profile assessment, then the method returns to step 820. If all data has been stored, then the method ends 860. In some embodiments, the processor determines if unstored data exists by determining if there is a row of data in the genotyping data below the just processed row.

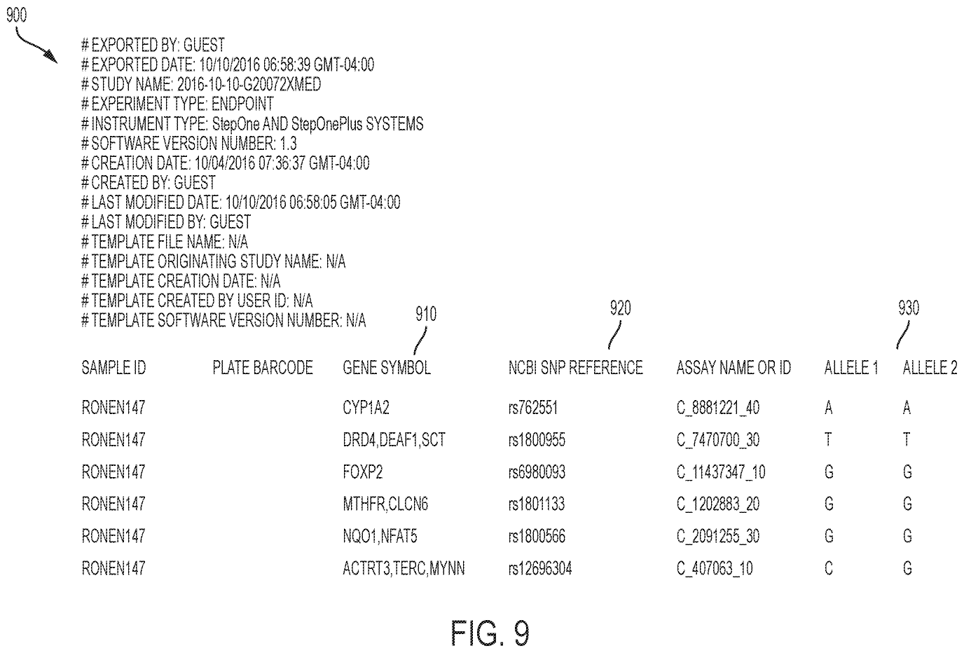

FIG. 9 shows exemplary genotyping data 900. Genotyping data may take the form of a text file saved by a user, wherein the text file is generated manually or as output from equipment for performing genotyping measurements (e.g. TaqMan™ SNP genotyping assays). FIG. 9 comprises 6 rows of genotyping data from a single biological sample (“RONEN147”). Each row corresponds to data for a different SNP. Each SNP of the genotyping data 900 is identified by at least a gene identifier 910 and a SNP reference 920. The gene identifier identifies the gene with which the SNP is associated. In certain embodiments, multiple (e.g. two or more) genes are associated with the SNP (e.g. the SNP may occur nearby two or more genes and influence phenotypes associated with each of the associated genes), and, accordingly, two or more corresponding gene identifiers are listed. Each SNP in the genotyping data has a corresponding variant identified by the allele measurements 930. The measurements “allele 1” and “allele 2” for a given SNP may be compared with measurement outcomes associated with the variants of a SNP object corresponding to the given SNP to populate an individual's personal genetic profile assessment.

The genotyping data in FIG. 9 used to populate an individual's personal genetic profile assessment is generated from one or more biological samples of the individual. However, the one or more biological samples used in populating an individual's personal genetic profile assessment may also be taken from a different human or a non-human animal. In some embodiments, genotyping data is generated from one or more biological samples of a non-human animal. For example, an individual may supply biological samples of their pet in order to understand genomic information about the pet to assist in providing better care. The animal may be a pet or may be an animal cared for by an individual. For example, the individual may be a veterinarian or a caretaker at a zoo charged with caring for the animal. In some embodiments, genotyping data is generated from one or more biological samples of a ward to whom the individual is a guardian. For example, a parent may supply one or more biological samples to genotyping data for their child in order to improve his/her childrearing.

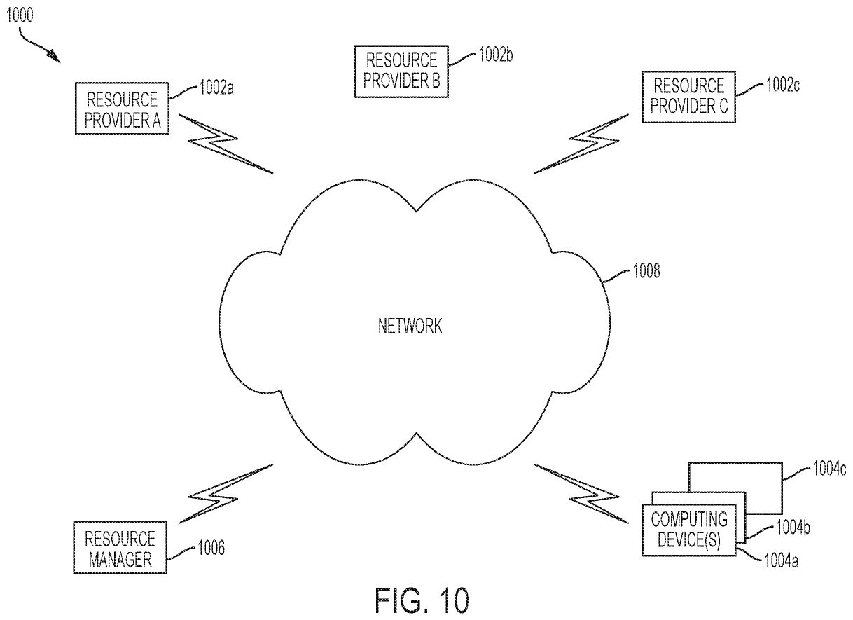

As shown in FIG. 10, an implementation of a network environment 1000 for use in providing systems and methods for creation of personal genetic profile products and assessments as described herein is shown and described. FIG. 10 shows an illustrative network environment 1000 for use in the methods and systems described herein. In brief overview, referring now to FIG. 10, a block diagram of an exemplary cloud computing environment 1000 is shown and described. The cloud computing environment 1000 may include one or more resource providers 1002a, 1002b, 1002c (collectively, 1002). Each resource provider 1002 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1002 may be connected to any other resource provider 1002 in the cloud computing environment 1000. In some implementations, the resource providers 1002 may be connected over a computer network 1008. Each resource provider 1002 may be connected to one or more computing device 1004a, 1004b, 1004c (collectively, 1004), over the computer network 1008.

The cloud computing environment 1000 may include a resource manager 1006. The resource manager 1006 may be connected to the resource providers 1002 and the computing devices 1004 over the computer network 1008. In some implementations, the resource manager 1006 may facilitate the provision of computing resources by one or more resource providers 1002 to one or more computing devices 1004. The resource manager 1006 may receive a request for a computing resource from a particular computing device 1004. The resource manager 1006 may identify one or more resource providers 1002 capable of providing the computing resource requested by the computing device 1004. The resource manager 1006 may select a resource provider 1002 to provide the computing resource. The resource manager 1006 may facilitate a connection between the resource provider 1002 and a particular computing device 1004. In some implementations, the resource manager 1006 may establish a connection between a particular resource provider 1002 and a particular computing device 1004. In some implementations, the resource manager 1006 may redirect a particular computing device 1004 to a particular resource provider 1002 with the requested computing resource.

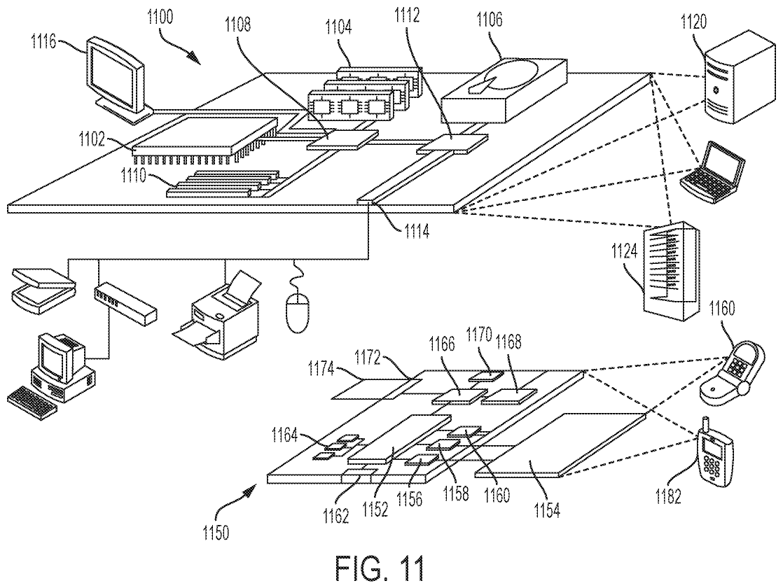

FIG. 11 shows an example of a computing device 1100 and a mobile computing device 1150 that can be used in the methods and systems described in this disclosure. The computing device 1100 is intended to represent various forms of digital computers, such as laptops, desktops, workstations, personal digital assistants, servers, blade servers, mainframes, and other appropriate computers. The mobile computing device 1150 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1100 includes a processor 1102, a memory 1104, a storage device 1106, a high-speed interface 1108 connecting to the memory 1104 and multiple high-speed expansion ports 1110, and a low-speed interface 1112 connecting to a low-speed expansion port 1114 and the storage device 1106. Each of the processor 1102, the memory 1104, the storage device 1106, the high-speed interface 1108, the high-speed expansion ports 1110, and the low-speed interface 1112, are interconnected using various busses, and may be mounted on a common motherboard or in other manners as appropriate. The processor 1102 can process instructions for execution within the computing device 1100, including instructions stored in the memory 1104 or on the storage device 1106 to display graphical information for a GUI on an external input/output device, such as a display 1116 coupled to the high-speed interface 1108. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1104 stores information within the computing device 1100. In some implementations, the memory 1104 is a volatile memory unit or units. In some implementations, the memory 1104 is a non-volatile memory unit or units. The memory 1104 may also be another form of computer-readable medium, such as a magnetic or optical disk.

The storage device 1106 is capable of providing mass storage for the computing device 1100. In some implementations, the storage device 1106 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1102), perform one or more methods, such as those described above. The instructions can also be stored by one or more storage devices such as computer- or machine-readable mediums (for example, the memory 1104, the storage device 1106, or memory on the processor 1102).

The high-speed interface 1108 manages bandwidth-intensive operations for the computing device 1100, while the low-speed interface 1112 manages lower bandwidth-intensive operations. Such allocation of functions is an example only. In some implementations, the high-speed interface 1108 is coupled to the memory 1104, the display 1116 (e.g., through a graphics processor or accelerator), and to the high-speed expansion ports 1110, which may accept various expansion cards (not shown). In the implementation, the low-speed interface 1112 is coupled to the storage device 1106 and the low-speed expansion port 1114. The low-speed expansion port 1114, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1100 may be implemented in a number of different forms, as shown in the figure. For example, it may be implemented as a standard server 1120, or multiple times in a group of such servers. In addition, it may be implemented in a personal computer such as a laptop computer 1122. It may also be implemented as part of a rack server system 1124. Alternatively, components from the computing device 1100 may be combined with other components in a mobile device (not shown), such as a mobile computing device 1150. Each of such devices may contain one or more of the computing device 1100 and the mobile computing device 1150, and an entire system may be made up of multiple computing devices communicating with each other.

The mobile computing device 1150 includes a processor 1152, a memory 1164, an input/output device such as a display 1154, a communication interface 1166, and a transceiver 1168, among other components. The mobile computing device 1150 may also be provided with a storage device, such as a micro-drive or other device, to provide additional storage. Each of the processor 1152, the memory 1164, the display 1154, the communication interface 1166, and the transceiver 1168, are interconnected using various buses, and several of the components may be mounted on a common motherboard or in other manners as appropriate.

The processor 1152 can execute instructions within the mobile computing device 1150, including instructions stored in the memory 1164. The processor 1152 may be implemented as a chipset of chips that include separate and multiple analog and digital processors. The processor 1152 may provide, for example, for coordination of the other components of the mobile computing device 1150, such as control of user interfaces, applications run by the mobile computing device 1150, and wireless communication by the mobile computing device 1150.

The processor 1152 may communicate with a user through a control interface 1158 and a display interface 1156 coupled to the display 1154. The display 1154 may be, for example, a TFT (Thin-Film-Transistor Liquid Crystal Display) display or an OLED (Organic Light Emitting Diode) display, or other appropriate display technology. The display interface 1156 may comprise appropriate circuitry for driving the display 1154 to present graphical and other information to a user. The control interface 1158 may receive commands from a user and convert them for submission to the processor 1152. In addition, an external interface 1162 may provide communication with the processor 1152, so as to enable near area communication of the mobile computing device 1150 with other devices. The external interface 1162 may provide, for example, for wired communication in some implementations, or for wireless communication in other implementations, and multiple interfaces may also be used.

The memory 1164 stores information within the mobile computing device 1150. The memory 1164 can be implemented as one or more of a computer-readable medium or media, a volatile memory unit or units, or a non-volatile memory unit or units. An expansion memory 1174 may also be provided and connected to the mobile computing device 1150 through an expansion interface 1172, which may include, for example, a SIMM (Single In Line Memory Module) card interface. The expansion memory 1174 may provide extra storage space for the mobile computing device 1150, or may also store applications or other information for the mobile computing device 1150. Specifically, the expansion memory 1174 may include instructions to carry out or supplement the processes described above, and may include secure information also. Thus, for example, the expansion memory 1174 may be provided as a security module for the mobile computing device 1150, and may be programmed with instructions that permit secure use of the mobile computing device 1150. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier and, when executed by one or more processing devices (for example, processor 1152), perform one or more methods, such as those described above. The instructions can also be stored by one or more storage devices, such as one or more computer- or machine-readable mediums (for example, the memory 1164, the expansion memory 1174, or memory on the processor 1152). In some implementations, the instructions can be received in a propagated signal, for example, over the transceiver 1168 or the external interface 1162.

The mobile computing device 1150 may communicate wirelessly through the communication interface 1166, which may include digital signal processing circuitry where necessary. The communication interface 1166 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1168 using a radio-frequency. In addition, short-range communication may occur, such as using a Bluetooth®, Wi-Fi™, or other such transceiver (not shown). In addition, a GPS (Global Positioning System) receiver module 1170 may provide additional navigation- and location-related wireless data to the mobile computing device 1150, which may be used as appropriate by applications running on the mobile computing device 1150.

The mobile computing device 1150 may also communicate audibly using an audio codec 1160, which may receive spoken information from a user and convert it to usable digital information. The audio codec 1160 may likewise generate audible sound for a user, such as through a speaker, e.g., in a handset of the mobile computing device 1150. Such sound may include sound from voice telephone calls, may include recorded sound (e.g., voice messages, music files, etc.) and may also include sound generated by applications operating on the mobile computing device 1150.

The mobile computing device 1150 may be implemented in a number of different forms, as shown in the figure. For example, it may be implemented as a cellular telephone 1180. It may also be implemented as part of a smart-phone 1182, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the data structures (e.g. products, categories, SNP objects, gene objects) described herein can be separated, combined or incorporated into single or combined data structures. The data structures depicted in the figures are not intended to limit the systems described herein to the data structure frameworks shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein. In view of the structure, functions and apparatus of the systems and methods described here, in some implementations.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method for creating personal genetic profile products that correspond to an assessment of an individual's genetic profile via a graphical user interface (GUI), the method comprising:
(a) presenting, by a processor of a computing device, a graphical user interface element for creation of a gene object corresponding to a specific gene, wherein the gene object is a data structure comprising:
one or more SNP objects, each SNP object corresponding to a specific SNP occurring in or nearby the specific gene with which the gene object is associated, and each SNP object comprising:
(i) a SNP reference; and
(ii) for each of one or more variants of the specific SNP to which the SNP object corresponds:
a measurement outcome associated with the variant;
a qualifier associated with the variant; and
optionally, additional information associated with the variant; and
(iii) optionally, additional information associated with the SNP object; and
(b) receiving, by the processor, via the graphical user interface element, a SNP object associated with the gene object;
(c) associating, by the processor, the received SNP object with the gene object; and
(d) storing, by the processor, the gene object for further retrieval and/or updating by the processor via the GUI.

2. The method of claim 1, wherein the gene object comprises a gene identifier that identifies the specific gene with which the gene object is associated.

3. The method of any one of claims 1 to 2, wherein the SNP reference is an alphanumeric code that serves as a unique identifier of the specific SNP with which the SNP object is associated.

4. The method of any one of claims 1 to 3, wherein, for each variant of the specific SNP to which the SNP object corresponds, the measurement outcome associated with the variant corresponds to an identification of a result of a genotyping measurement of the specific SNP with which the SNP object that comprises the variant is associated.

5. The method of claim 4, wherein the measurement outcome associated with the variant comprises an identification of a first nucleotide associated with a first copy of an individual's genetic material, and an identification of a second nucleotide of a second copy of an individual's genetic material.

6. The method of any one of claims 1 to 5, wherein the SNP object comprises, for each of three physically viable variants of the specific SNP to which the SNP object corresponds:
a measurement outcome associated with the variant; and
a qualifier associated with the variant.

7. The method of any one of claims 1 to 6, wherein, for each variant of the specific SNP to which the SNP object corresponds, the qualifier associated with the variant corresponds to at least one of (i) and (ii) as follows:
(i) a classification of the variant based on prevalence of the variant within a population; and
(ii) a classification of the variant based on a health-related phenotype associated with the variant.

8. The method of any one of claims 1 to 7, wherein, for each variant of the specific SNP to which the SNP object corresponds, the particular qualifier associated with the variant is a specific qualifier of a countable set of predefined qualifiers.

9. The method of any one of claims 1 to 8, wherein the SNP object comprises additional information associated with the SNP object, the additional information comprising at least one of:
(i) a short description of the specific SNP to which the SNP object corresponds;
(ii) a description; and
(iii) one or more references, each reference identifying a source of information.

10. The method of any one of claims 1 to 9, wherein the SNP object comprises, for each variant of the specific SNP to which the SNP object corresponds, additional information associated with the variant, the additional information comprising a description of the variant.

11. The method of any one of claims 1 to 10, comprising receiving a user input to initiate creation of the gene object.

12. The method of any one of claims 1 to 11, wherein receiving the SNP object associated with the gene object comprises receiving, by the processor, via the graphical user interface element, a user input of:
(i) a SNP reference; and
(ii) for each of one or more variants, a measurement outcome associated with the variant and a qualifier associated with the variant.

13. The method of any one of claims 1 to 12, wherein:
step (a) comprises:
presenting a SNP reference graphical control element for user entry of a SNP reference; and
for each of one or more variants to be represented in a SNP object, presenting:
(i) a respective variant measurement graphical control element for user entry of a measurement outcome associated with the respective variant; and
(ii) a respective variant qualifier graphical control element for user selection of a qualifier associated with the respective variant, and
step (b) comprises:
receiving, via the SNP reference graphical control element, a user input of a SNP reference; and
for each of the one or more variants to be represented in the SNP object:
(i) receiving, via the respective variant measurement graphical control element, a user input of a measurement outcome; and
(ii) receiving, via the respective variant qualifier graphical control element, a user selection of a qualifier.

14. The method of any one of claims 1 to 13, comprising:
(e) presenting, by the processor, a graphical user interface element for creation of a category corresponding to a health-related phenotype associated with a predefined group of one or more related SNPs each of which is associated with the health-related phenotype, wherein the category is a data structure comprising one or more SNP objects;
(f) receiving, by the processor, via the graphical user interface element for creation of a category, a selection of one or more SNP objects;
(g) associating, by the processor, the one or more selected SNP objects with the category; and
(h) storing, by the processor, the category, for further retrieval and/or updating by the processor via the GUI.

15. The method of claim 14, wherein the category comprises additional information selected from the group consisting of:
(i) a name of the category;
(ii) a background image associated with the category;
(iii) an icon associated with the category;
(iv) a category order identifier associated with the category; and
(v) a description of the category.

16. The method of any one of claims 14 to 15 wherein:
step (e) comprises presenting (i) a first gene selection graphical control element for selecting a first gene object and (ii) a first SNP selection graphical control element for selecting a first SNP object of the first gene object, and
step (f) comprises receiving, via the first gene selection graphical control element, a user selection of a first gene object, and receiving, via the first SNP selection graphical control element, a user selection of a first SNP object of the selected first gene object.

17. The method of claim 16, wherein
the first gene selection graphical control element displays a list of selectable elements, each element of the list corresponding to a previously stored gene object, and
following a user selection of the first gene object, the first SNP selection graphical control element displays a list of selectable elements, each element of the list corresponding to a SNP object of the previously selected first gene object.

18. The method of any one of claim 16 or 17, comprising:
receiving, by the processor, a user input to add more genes to the category; and
responsive to the user input to add more genes to the category, presenting (i) a second gene selection graphical control element for selecting a second gene object and (ii) a second SNP selection graphical control element for selecting a second SNP object of the second gene object.

19. The method of claims 14 to 18, comprising:
(i) presenting, by the processor, a graphical user interface element for creation of a product corresponding to a general class of health-related phenotypes, e.g. fitness, e.g. nutritional health, e.g. exceptional qualities associated with a predefined group of one or more related categories, wherein the product is a data structure comprising one or more categories;
(j) receiving, by the processor, via the graphical user interface element for creation of the product, a selection of one or more categories;
(k) associating, by the processor, the one or more selected categories with the product; and
(l) storing, by the processor, the product for further retrieval and/or updating by the GUI.

20. The method of claim 18, wherein the product comprises additional information selected from the group consisting of:
(i) a name of the product;
(ii) an icon associated with the product; and
(iii) a description of the product.

21. A method of creating and/or updating personal genetic profile assessments for a plurality of individuals, the method comprising:
receiving, by a processor of a computing device, genotyping data collected from a plurality of biological samples, each biological sample having been taken from a particular individual of the plurality of individuals, wherein the genotyping data comprises, for each biological sample, one or more genotyping measurements of one or more SNPs, each SNP associated with one or more genes; and
for each individual associated with each biological sample, for each SNP:
(a) identifying, by the processor, a gene object corresponding to a target gene of the one or more genes with which the SNP is associated and a SNP object of the gene object corresponding to the SNP;
(b) receiving, by the processor, a determination of a particular variant of the SNP that the individual has and determining a qualifier associated with the particular variant; and
(c) storing, by the processor, within the personal genetic profile assessment of the individual:
(i) a gene identifier associated with the measured SNP, wherein the gene identifier identifies the target gene;
(ii) a SNP reference that identifies the measured SNP, wherein the SNP reference is associated with the gene identifier;
(iii) a measurement outcome that identifies the particular variant of the SNP that the individual has, wherein the measurement outcome is associated with the SNP reference; and
(iv) the qualifier associated with the particular variant, wherein the qualifier is associated with the SNP reference.

22. The method of claim 21, wherein, for each biological sample, the one or more genotyping measurements of the one or more SNPs comprise(s), for each SNP, a first measurement that identifies a first nucleotide of a first copy (of an individual's genetic material, and a second measurement that identifies a second nucleotide of a second copy of an individual's genetic material.

23. The method of any one of claims 21 to 22, wherein the genotyping data comprises data from a PCR-based SNP genotyping assay.

24. The method of any one of claims 21 to 23, comprising measuring, via a PCR-based SNP genotyping assay, one or more SNPs for a plurality of biological samples, thereby producing genotyping data.

25. The method of any one of claims 21 to 24,
wherein for each biological sample:
the genotyping data comprises, for each gene with which each SNP is associated, a corresponding gene identifier, and
the genotyping data comprises, for each SNP, a corresponding SNP reference, and
wherein step (a) comprises, for each measured SNP:
accessing, by the processor, a plurality of previously stored gene objects, wherein each previously stored gene object is a data structure comprising a gene identifier and one or more SNP objects, each SNP object of a previously stored gene object comprising a SNP reference;
matching, by the processor, a gene identifier of the genotyping data to a gene identifier of one of the plurality of previously stored gene objects, thereby identifying the gene object associated with the gene to which the SNP belongs; and
matching, by the processor, the SNP reference (corresponding to the measured SNP) of the genotyping data to a SNP reference of one of the SNP objects of the plurality of previously stored gene objects, thereby identifying the SNP object of the gene object corresponding to the SNP,
thereby identifying the gene object corresponding to the target gene with which the SNP is associated and the SNP object corresponding to the SNP.

26. The method of any one of claims 21 to 25, wherein:
the genotyping data comprises, for each SNP, a corresponding SNP reference, and
for each biological sample, for each measured SNP, determining, by the processor, a particular variant of the SNP that the individual associated with the biological sample has and a qualifier associated with the variant comprises:
accessing, by the processor, a plurality of previously stored gene objects (e.g. gene objects previously created and stored via the methods of any one of claims 1 to 13), wherein each gene object comprises:
one or more SNP objects, each SNP object comprising:
a SNP reference; and
for each of one or more variants of a specific SNP associated with the SNP object, a measurement outcome associated with the variant, and a qualifier associated with the variant;
matching, by the processor, the SNP reference (corresponding to the measured SNP) of the genotyping data to a SNP reference of one of the SNP objects of the plurality of previously stored gene objects, thereby identifying a SNP object corresponding to the measured SNP; and
matching, by the processor, the genotyping measurement of the measured SNP to a measurement outcome of identified SNP object corresponding to the measured SNP, thereby determining the particular variant of the SNP that the individual has.

27. The method of any one of claims 21 to 26, comprising automatically determining, by the processor, for each individual of the plurality of individuals associated with the plurality of measured biological samples, one or more products associated with the individual's personal genetic profile assessment, wherein:
each product corresponds to a predefined group of one or more related categories, and the product is a data structure comprising one or more categories,
each category corresponds to a predefined group of one or more related SNPs, each of which is associated with a health-related phenotype, wherein the category is a data structure comprising one or more SNP objects, and
for each individual of the plurality of individuals, automatically determining one or more products associated with the individual's personal genetic profile assessment comprises, for each product of a plurality of previously stored products:
determining a list of all the different SNP objects that the product comprises, the list comprising each distinct SNP object of each category the product comprises; and
matching, by the processor, the list of all the different SNP objects of the product to the measured SNPs of the genotyping data collected from the biological sample associated with the individual.

28. The method of any one of claims 21 to 27, comprising, for an individual of the plurality of individuals, causing, by the processor, display of an assessment graphical user interface (GUI) for view of the individual's personal genetic profile assessment, the assessment GUI comprising, for each measured SNP associated with the individual, a graphical element comprising:
(A) at least one of (i) or (ii) as follows:
(i) a graphical representation of the associated (with the measured SNP) gene identifier of the individual's personal genetic profile assessment; and
(ii) a graphical representation of the SNP reference of the individual's personal genetic profile assessment that identifies the measured SNP; and
(B) a graphical representation of the qualifier of the individual's personal genetic profile assessment, wherein the qualifier is the qualifier that is associated with the SNP reference that identifies the measured SNP.

29. The method of claim 28, wherein the causing of the assessment graphical user interface (GUI) for view of the individual's personal genetic profile assessment comprises:
causing, by the processor, display of one or more selectable product graphical control elements, each selectable product graphical control element corresponding to a previously stored product, wherein:
each previously stored product corresponds to a predefined group of one or more related categories, and
each category corresponds to a predefined group of one or more related gene-SNP combinations each of which is associated with the health-related phenotype;
responsive to a user selection of a specific product graphical control element, for each category of the previously stored product corresponding to the selected product graphical control element, causing display of a respective selectable category graphical control element; and
responsive to a user selection of a specific category graphical control element, for each gene-SNP combination of the corresponding category, causing display of a corresponding gene graphical control element, wherein each gene graphical control element comprises:
at least one of (i) or (ii) as follows:
(i) a graphical representation of the associated (with the measured SNP) gene identifier of the individual's personal genetic profile assessment; and
(ii) a graphical representation of the SNP reference of the individual's personal genetic profile assessment that identifiers the measured SNP; and
a graphical representation of the qualifier of the individual's personal genetic profile assessment, wherein the qualifier is the qualifier that is associated with the SNP reference that identifies the measured SNP.

30. The method of any one of claims 21 to 29, comprising:
receiving, by the processor, a command to share data corresponding to at least a portion of the personal genetic profile assessment of an individual of the plurality of individuals;
receiving, by the processor, an identification of a sharing entity with which to share the data corresponding to the genetic profile assessment of the individual; and
responsive to receipt of the command to share data and the identification sharing entity, providing, by the processor, the data corresponding to the at least a portion of the personal genetic profile assessment of the individual to the sharing entity.

31. The method of claim 30, wherein the command to share data corresponding to the at least a portion of the personal genetic profile assessment of the individual comprises an identification of a shareable portion (up to all) of the measured SNPs associated with the individual and the data provided to the sharing entity includes only data corresponding each measured SNP of the shareable portion of the measured SNPs associated with the individual.

32. The method of any one of claim 30 or 31, wherein the data corresponding to the at least a portion of the individual's personal genetic profile assessment comprises, for each measured SNP of a shareable portion (up to all) of the measured SNPs associated with the individual:
at least one of:
(i) the gene identifier associated with the measured SNP; and
(ii) the SNP reference associated with the measured SNP; and
at least one of:
(iii) the particular variant of the SNP that the individual has; and
(iv) the qualifier associated with the particular variant of the SNP that the individual has.

33. The method of any one of claims 30 to 32, wherein providing the data corresponding to the portion of the personal genetic profile assessment of the individual to the sharing entity comprises:
generating an assessment report comprising, for each measured SNP of a shareable portion (up to all) of the measured SNPs associated with the individual:
a first graphical representation corresponding to a graphical representation of at least one of:
(i) the gene identifier associated with the measured SNP; and
(ii) the SNP reference associated with the measured SNP; and
in relation (e.g., in spatial proximity to, such as in a same row of a table) to the first graphical representation, a second graphical representation corresponding to a graphical representation of at least one of:
(iii) the particular variant of the SNP that the individual has; and
(iv) the qualifier associated with the particular variant of the SNP that the individual has; and
providing, by the processor, the assessment report to the sharing entity (e.g., sending via email, text message, a web link from which the assessment report can be downloaded).

34. The method of any one of claims 30 to 33, wherein the data corresponding to the at least a portion of the personal genetic profile assessment of the individual comprises an identification a shareable portion (up to all) of the measured SNPs associated with the individual, each measured SNP of the shared portion corresponding to a shareable SNP, and wherein providing the data corresponding to the portion of the personal genetic profile assessment of the individual to the sharing entity comprises causing, by the processor, display of an assessment graphical user interface (GUI) for view of the portion of the personal genetic profile assessment of the individual, the assessment GUI comprising, for each shareable SNP, a graphical element comprising:
(A) at least one of (i) or (ii) as follows:
(i) a graphical representation of the associated (with the shareable SNP) gene identifier of the personal genetic profile assessment of the individual;
(ii) a graphical representation of the SNP reference of the personal genetic profile assessment of the individual, wherein the SNP reference identifies the shareable SNP; and
(B) a graphical representation of the qualifier of the personal genetic profile assessment individual, wherein the qualifier is the qualifier that is associated with the SNP reference that identifies the shareable SNP.

35. The method of claim 34, comprising:
causing, by the processor, display of one or more selectable product graphical control elements, each selectable product graphical control element corresponding to a previously stored product, wherein:
each previously stored product corresponds to a predefined group of one or more related categories, and
each category corresponds to a predefined group of one or more related gene-SNP combinations each of which is associated with the health-related phenotype;
responsive to a user selection of a specific product graphical control element, for each category of the previously stored product corresponding to the selected product graphical control element, causing display of a respective selectable category graphical control element; and
responsive to a user selection of a specific category graphical control element, for each gene-SNP combination of the corresponding category and corresponding to a shareable SNP, causing display of a corresponding gene graphical control element, wherein each gene graphical control element comprises:
at least one of (i) or (ii) as follows:
(i) a graphical representation of the associated (with the shareable SNP) gene identifier of the individual's personal genetic profile assessment;
(ii) a graphical representation of the SNP reference of the individual's personal genetic profile assessment that identifiers the shareable SNP; and
a graphical representation of the qualifier of the individual's personal genetic profile assessment, wherein the qualifier is the qualifier that is associated with the SNP reference that identifies the shareable SNP.

36. A system for creating personal genetic profile products that correspond to an assessment of an individual's genetic profile via a graphical user interface (GUI), the system comprising:
a processor; and
a memory having instructions stored thereon, wherein the instructions, when executed by the processor, cause the processor to:
(a) present a graphical user interface element for creation of a gene object corresponding to a specific gene, wherein the gene object is a data structure comprising:
one or more SNP objects, each SNP object corresponding to a specific SNP occurring in or nearby the specific gene with which the gene object is associated, and each SNP object comprising:
(i) a SNP reference; and
(ii) for each of one or more variants of the specific SNP to which the SNP object corresponds:
a measurement outcome associated with the variant;
a qualifier associated with the variant; and
optionally, additional information associated with the variant; and
(iii) optionally, additional information associated with the SNP object; and
(b) receive, via the graphical user interface element, a SNP object associated with the gene object;
(c) associate the received SNP object with the gene object; and
(d) store the gene object for further retrieval and/or updating by the processor via the GUI.

37. The system of claim 36, wherein the gene object comprises a gene identifier that identifies the specific gene with which the gene object is associated.

38. The system of any one of claims 36 to 37, wherein the SNP reference is an alphanumeric code that serves as a unique identifier of the specific SNP with which the SNP object is associated.

39. The system of any one of claims 36 to 38, wherein, for each variant of the specific SNP to which the SNP object corresponds, the measurement outcome associated with the variant corresponds to an identification of a result of a genotyping measurement of the specific SNP with which the SNP object that comprises the variant is associated.

40. The system of claim 39, wherein the measurement outcome associated with the variant comprises an identification of a first nucleotide associated with a first copy of an individual's genetic material, and an identification of a second nucleotide of a second copy of an individual's genetic material.

41. The system of any one of claims 36 to 40, wherein the SNP object comprises, for each of three physically viable variants of the specific SNP to which the SNP object corresponds:
a measurement outcome associated with the variant; and
a qualifier associated with the variant.

42. The system of any one of claims 36 to 41, wherein, for each variant of the specific SNP to which the SNP object corresponds, the qualifier associated with the variant corresponds to at least one of (i) and (ii) as follows:
(i) a classification of the variant based on prevalence of the variant within a population; and
(ii) a classification of the variant based on a health-related phenotype associated with the variant.

43. The system of any one of claims 36 to 41, wherein, for each variant of the specific SNP to which the SNP object corresponds, the particular qualifier associated with the variant is a specific qualifier of a countable set of predefined qualifiers.

44. The system of any one of claims 36 to 43, wherein the SNP object comprises additional information associated with the SNP object, the additional information comprising at least one of:
(i) a short description of the specific SNP to which the SNP object corresponds;
(ii) a description; and
(iii) one or more references, each reference identifying a source of information.

45. The system of any one of claims 36 to 44, wherein the SNP object comprises, for each variant of the specific SNP to which the SNP object corresponds, additional information associated with the variant, the additional information comprising a description of the variant.

46. The system of any one of claims 36 to 45, wherein the instructions cause the processor to receive a user input to initiate creation of the gene object.

47. The system of any one of claims 36 to 46, wherein the instructions cause the processor to receive the SNP object associated with the gene object by receiving, via the graphical user interface element, a user input of:
(i) a SNP reference; and
(ii) for each of one or more variants, a measurement outcome associated with the variant and a qualifier associated with the variant.

48. The system of any one of claims 36 to 47, wherein the instructions cause the processor to:
at step (a):
present a SNP reference graphical control element for user entry of a SNP reference; and
for each of one or more variants to represented in a SNP object, present:
(i) a respective variant measurement graphical control element for user entry of a measurement outcome associated with the respective variant; and
(ii) a respective variant qualifier graphical control element for user selection of a qualifier associated with the respective variant; and
at step (b):
receive, via the SNP reference graphical control element, a user input of a SNP reference; and
for each of the one or more variants to be represented in the SNP object:
(i) receive, via the respective variant measurement graphical control element, a user input of a measurement outcome; and
(ii) receive, via the respective variant qualifier graphical control element, a user selection of a qualifier.

49. The system of any one of claims 36 to 48, wherein the instructions cause the processor to:
(e) present a graphical user interface element for creation of a category corresponding to a health-related phenotype associated with a predefined group of one or more related SNPs each of which is associated with the health-related phenotype, wherein the category is a data structure comprising one or more SNP objects;
(f) receive, via the graphical user interface element for creation of a category, a selection of one or more SNP objects;
(g) associate the one or more selected SNP objects with the category; and
(h) store the category for further retrieval and/or updating by the processor via the GUI.

50. The system of claim 49, wherein the category comprises additional information selected from the group consisting of:
(i) a name of the category;
(ii) a background image associated with the category;
(iii) an icon associated with the category;
(iv) a category order identifier associated with the category; and
(v) a description of the category.

51. The system of any one of claim 49 or 50 wherein the instructions cause the processor to:
at step (e), present (i) a first gene selection graphical control element for selecting a first gene object and (ii) a first SNP selection graphical control element for selecting a first SNP object of the first gene object; and
at step (f):
receive, via the first gene selection graphical control element, a user selection of a first gene object; and
receive, via the first SNP selection graphical control element, a user selection of a first SNP object of the selected first gene object.

52. The system of claim 51, wherein
the first gene selection graphical control element displays a list of selectable elements, each element of the list corresponding to a previously stored gene object, and
following a user selection of the first gene object, the first SNP selection graphical control element displays a list of selectable elements, each element of the list corresponding to a SNP object of the previously selected first gene object.

53. The system of any one of claim 51 or 52, wherein the instructions cause the processor to:
receive a user input to add more genes to the category; and
responsive to the user input to add more genes to the category, present (i) a second gene selection graphical control element for selecting a second gene object and (ii) a second SNP selection graphical control element for selecting a second SNP object of the second gene object.

54. The system of any one of claims 49 to 53, wherein the instructions cause the processor to:
(i) present a graphical user interface element for creation of a product corresponding to a general class of health-related phenotypes associated with a predefined group of one or more related categories, wherein the product is a data structure comprising one or more categories;
(j) receive, via the graphical user interface element for creation of the product, a selection of one or more categories;
(k) associate the one or more selected categories with the product; and
(l) store the product for further retrieval and/or updating by the GUI.

55. The system of claim 54, wherein the product comprises additional information selected from the group consisting of:
(i) a name of the product;
(ii) an icon associated with the product; and
(iii) a description of the product.

56. A system for creating and/or updating personal genetic profile assessments for a plurality of individuals, the system comprising:
a processor; and
a memory having instructions stored thereon, wherein the instructions, when executed by the processor, cause the processor to:
receive genotyping data collected from a plurality of biological samples, each biological sample having been taken from a particular individual of the plurality of individuals, wherein the genotyping data comprises, for each biological sample, one or more genotyping measurements of one or more SNPs, each SNP associated with one or more genes; and
for each individual associated with each biological sample, for each SNP:
(a) identify a gene object corresponding to a target gene of the one or more genes with which the SNP is associated and a SNP object of the gene object corresponding to the SNP;
(b) receive a determination of a particular variant of the SNP that the individual has and determining a qualifier associated with the particular variant; and
(c) store within the personal genetic profile assessment of the individual:
(i) a gene identifier associated with the measured SNP, wherein the gene identifier identifies the target gene;
(ii) a SNP reference that identifies the measured SNP, wherein the SNP reference is associated with the gene identifier;
(iii) a measurement outcome that identifies the particular variant of the SNP that the individual has, wherein the measurement outcome is associated with the SNP reference; and
(iv) the qualifier associated with the particular variant, wherein the qualifier is associated with the SNP reference.

57. The system of claim 56, wherein, for each biological sample, the one or more genotyping measurements of the one or more SNPs comprise(s), for each SNP, a first measurement that identifies a first nucleotide of a first copy of an individual's genetic material, and a second measurement that identifies a second nucleotide of a second copy of an individual's genetic material.

58. The system of any one of claims 56 to 57, wherein the genotyping data comprises data from a PCR-based SNP genotyping assay.

59. The system of any one of claims 56 to 58, comprising a reader for measuring genotyping data for one or more SNPs for a plurality of biological samples and providing the genotyping data to the processor.

60. The system of any one of claims 56 to 59, wherein,
for each biological sample:
the genotyping data comprises, for each gene with which each SNP is associated, a corresponding gene identifier, and
the genotyping data comprises, for each SNP, a corresponding SNP reference, and
wherein the instructions cause the processor to, at step (a), for each measured SNP:
access a plurality of previously stored gene objects, wherein each previously stored gene object is a data structure comprising a gene identifier and one or more SNP objects, each SNP object of a previously stored gene object comprising a SNP reference;
match a gene identifier of the genotyping data to a gene identifier of one of the plurality of previously stored gene objects, thereby identifying the gene object associated with the gene to which the SNP belongs; and
match the SNP reference (corresponding to the measured SNP) of the genotyping data to a SNP reference of one of the SNP objects of the plurality of previously stored gene objects, thereby identifying the SNP object of the gene object corresponding to the SNP,
thereby identifying the gene object corresponding to the target gene with which the SNP is associated and the SNP object corresponding to the SNP.

61. The system of any one of claims 56 to 60, wherein:
the genotyping data comprises, for each SNP, a corresponding SNP reference, and
the instructions cause the processor to, for each biological sample, for each measured SNP, determine a particular variant of the SNP that the individual associated with the biological sample has and a qualifier associated with the variant by:
accessing a plurality of previously stored gene objects, wherein each gene object comprises:
one or more SNP objects, each SNP object comprising:
a SNP reference; and
for each of one or more variants of a specific SNP associated with the SNP object, a measurement outcome associated with the variant, and a qualifier associated with the variant;
matching the SNP reference (corresponding to the measured SNP) of the genotyping data to a SNP reference of one of the SNP objects of the plurality of previously stored gene objects, thereby identifying a SNP object corresponding to the measured SNP; and
matching the genotyping measurement of the measured SNP to a measurement outcome of identified SNP object corresponding to the measured SNP, thereby determining the particular variant of the SNP that the individual has.

62. The system of any one of claims 56 to 61, wherein the instructions cause the processor to, for each individual of the plurality of individuals associated with the plurality of measured biological samples, automatically determine one or more products associated with the individual's personal genetic profile assessment, wherein:
each product corresponds to a predefined group of one or more related categories, and the product is a data structure comprising one or more categories,
each category corresponds to a predefined group of one or more related SNPs, each of which is associated with a health-related phenotype, wherein the category is a data structure comprising one or more SNP objects, and
for each individual of the plurality of individuals, the instructions cause the processor to determine one or more products associated with the individual's personal genetic profile assessment by, for each product of a plurality of previously stored products:
determining a list of all the different SNP objects that the product comprises, the list comprising each distinct SNP object of each category the product comprises; and
matching the list of all the different SNP objects of the product to the measured SNPs of the genotyping data collected from the biological sample associated with the individual.

63. The system of any one of claims 56 to 62, wherein the instructions cause the processor to, for an individual of the plurality of individuals, cause display of an assessment graphical user interface (GUI) for view of the individual's personal genetic profile assessment, the assessment GUI comprising, for each measured SNP associated with the individual, a graphical element comprising:
(A) at least one of (i) or (ii) as follows:
(i) a graphical representation of the associated (with the measured SNP) gene identifier of the individual's personal genetic profile assessment; and
(ii) a graphical representation of the SNP reference of the individual's personal genetic profile assessment that identifies the measured SNP; and
(B) a graphical representation of the qualifier of the individual's personal genetic profile assessment, wherein the qualifier is the qualifier that is associated with the SNP reference that identifies the measured SNP.

64. The system of claim 63, wherein the instructions cause the processor to:
cause display of one or more selectable product graphical control elements, each selectable product graphical control element corresponding to a previously stored product, wherein:
each previously stored product corresponds to a predefined group of one or more related categories, and
each category corresponds to a predefined group of one or more related gene-SNP combinations each of which is associated with the health-related phenotype;
responsive to a user selection of a specific product graphical control element, for each category of the previously stored product corresponding to the selected product graphical control element, cause display of a respective selectable category graphical control element;
responsive to a user selection of a specific category graphical control element, for each gene-SNP combination of the corresponding category, cause display of a corresponding gene graphical control element, wherein each gene graphical control element comprises:
at least one of (i) or (ii) as follows:
(i) a graphical representation of the associated (with the measured SNP) gene identifier of the individual's personal genetic profile assessment; and
(ii) a graphical representation of the SNP reference of the individual's personal genetic profile assessment that identifiers the measured SNP; and
a graphical representation of the qualifier of the individual's personal genetic profile assessment, wherein the qualifier is the qualifier that is associated with the SNP reference that identifies the measured SNP.

65. The system of any one of claims 56 to 64, wherein the instructions cause the processor to:
receive a command to share data corresponding to at least a portion of the personal genetic profile assessment of an individual of the plurality of individuals;
receive an identification of a sharing entity with which to share the data corresponding to the genetic profile assessment of the individual; and
responsive to receipt of the command to share data and the identification sharing entity, provide the data corresponding to the at least a portion of the personal genetic profile assessment of the individual to the sharing entity.

66. The system of claim 65, wherein the command to share data corresponding to the at least a portion of the personal genetic profile assessment of the individual comprises an identification of a shareable portion (up to all) of the measured SNPs associated with the individual and the data provided to the sharing entity includes only data corresponding each measured SNP of the identified shareable portion of the measured SNPs associated with the individual.

67. The system of any one of claim 65 or 66, wherein the data corresponding to the at least a portion of the individual's personal genetic profile assessment comprises, for each measured SNP of a shareable portion (up to all) of the measured SNPs associated with the individual:
at least one of:
(i) the gene identifier associated with the measured SNP; and
(ii) the SNP reference associated with the measured SNP; and
at least one of:
(iii) the particular variant of the SNP that the individual has; and
(iv) the qualifier associated with the particular variant of the SNP that the individual has.

68. The system of any one of claims 65 to 67, wherein the instructions cause the processor to provide the data corresponding to the portion of the personal genetic profile assessment of the individual to the sharing entity by:
generating an assessment report comprising, for each measured SNP of a shareable portion (up to all) of the measured SNPs associated with the individual:
a first graphical representation corresponding to a graphical representation of at least one of:
(i) the gene identifier associated with the measured SNP; and
(ii) the SNP reference associated with the measured SNP; and
in relation to the first graphical representation, a second graphical representation corresponding to a graphical representation of at least one of:
(iii) the particular variant of the SNP that the individual has; and
(iv) the qualifier associated with the particular variant of the SNP that the individual has; and
providing the assessment report to the sharing entity.

69. The system of any one of claims 65 to 68, wherein the data corresponding to the at least a portion of the personal genetic profile assessment of the individual comprises an identification a shareable portion (up to all) of the measured SNPs associated with the individual, each measured SNP of the shareable portion corresponding to a shareable SNP, and wherein the instructions cause the processor to provide the data corresponding to the portion of the personal genetic profile assessment of the individual to the sharing entity by causing display of an assessment graphical user interface (GUI) for view of the portion of the personal genetic profile assessment of the individual, the assessment GUI comprising, for each shareable SNP, a graphical element comprising:
(A) at least one of (i) or (ii) as follows:
(i) a graphical representation of the associated (with the shareable SNP) gene identifier of the personal genetic profile assessment of the individual; and
(ii) a graphical representation of the SNP reference of the personal genetic profile assessment of the individual, wherein the SNP reference identifies the shareable SNP; and
(B) a graphical representation of the qualifier of the personal genetic profile assessment individual, wherein the qualifier is the qualifier that is associated with the SNP reference that identifies the shareable SNP.

70. The system of claim 69, wherein the instructions cause the processor to:
cause display of one or more selectable product graphical control elements, each selectable product graphical control element corresponding to a previously stored product, wherein:
each previously stored product corresponds to a predefined group of one or more related categories, and
each category corresponds to a predefined group of one or more related gene-SNP combinations each of which is associated with the health-related phenotype;
responsive to a user selection of a specific product graphical control element, for each category of the previously stored product corresponding to the selected product graphical control element, cause display of a respective selectable category graphical control element; and
responsive to a user selection of a specific category graphical control element, for each gene-SNP combination of the corresponding category and corresponding to a shareable SNP, cause display of a corresponding gene graphical control element, wherein each gene graphical control element comprises:
at least one of (i) or (ii) as follows:
(i) a graphical representation of the associated (with the shareable SNP) gene identifier of the individual's personal genetic profile assessment; and
(ii) a graphical representation of the SNP reference of the individual's personal genetic profile assessment that identifiers the shareable SNP; and
a graphical representation of the qualifier of the individual's personal genetic profile assessment, wherein the qualifier is the qualifier that is associated with the SNP reference that identifies the shareable SNP.
